(19) United States
(12) Patent Application Publication
(10) Pub. No.: US 2017/0273917 A1
(43) Pub. Date: Sep. 28, 2017

(54) NMDA RECEPTOR ANTAGONISTS FOR TREATING GAUCHER DISEASE

(71) Applicant: Yeda Research and Development Co. Ltd., Rehovot (IL)

(72) Inventors: Anthony FUTERMAN, Rehovot (IL); Andres David KLEIN, Rehovot (IL)

(21) Appl. No.: 15/511,033

(22) PCT Filed: Sep. 10, 2015
(86) PCT No.: PCT/IL2015/050931

§ 371 Date: Mar. 14, 2017

(30) Foreign Application Priority Data
Sep. 14, 2014 (IL) 234638

(51) Int. Cl. A61K 31/13; G01N 33/50; A61K 49/00; C12Q 1/68

(52) U.S. Cl. CPC A61K 31/13; C12Q 1/6883; G01N 33/5058; A61K 49/0004; G01N 33/50; C12Q 2600/178; G01N 2800/28; G01N 2800/2842; C12Q 2600/112; C12Q 2600/156

(57) ABSTRACT

The present invention provides pharmaceutical compositions comprising at least one NMDA receptor antagonist for use in treating neuropathic forms of Gaucher Disease (nGD).

### FIELD OF THE INVENTION

The present invention relates to compositions comprising NMDA receptor antagonists for use in the treatment of neuropathic forms of Gaucher Disease.

### BACKGROUND OF THE INVENTION

### Gaucher Disease

Lysosomal storage disorders (LSDs) encompass about 50 different inherited diseases. They are caused by deficiencies in lysosomal enzymes or transporters, resulting in intra-lysosomal accumulation of undegraded metabolites. While LSDs are individually rare, collectively they have a very high prevalence in the population, of about 1/7000 newborns (Fuller et al., 2006). This frequency is comparable to that of the most common genetic diseases (Walker, 2007; Ratjen and Doring, 2003).

Among the LSDs, Gaucher Disease (GD) is the most prevalent, with an overall frequency of 1 in 40,000, but in Ashkenazi Jews, as many as 1 in 500 are affected (Fuller et al., 2006). GD is caused by mutations in the GBA1 gene, which encodes β-glucocerebrosidase (also called acid β-glucosidase, D-glucosyl-N-acylsphingosine glucohydrolase, or GCase), a lysosome enzyme with glucosylceramidase activity that is needed to cleave, by hydrolysis, the beta-glucosidic linkage of glucosylceramide (GlcCer, also called glucocerebroside), an intermediate in glycolipid metabolism. As a consequence, cells accumulate large quantities of GlcCer, and eventually die.

From a clinical perspective, GD can be divided into three sub-types based on age of onset and on signs of nervous system involvement. The major symptoms of Type 1 are enlargement of spleen and liver, anaemia, thrombocytopenia, and skeletal lesions. Type 2 and 3, the neuropathic forms of GD (nGD), are classified according to the time of onset and rate of progression of neurological symptoms. Type 2, the acute neuropathic form, usually refers to children who display neurological abnormalities before 6 months of age and die by age 2-4 years of age. In Type 3, the sub-acute, chronic neuropathic form, patients present with similar symptoms to those observed in Type 2, but with a later onset and severity (Cox, 2010).

### Gaucher Disease Treatments

Type 1 patients can be treated with Enzyme Replacement Therapies (ERT). Treatments and drugs for GD Type 1, the most common form of the disease, may vary depending on the severity of each patient's disease and the course of treatment determined by a physician.

There are three ERTs available for treatment of GD and one oral medication which may be taken by those over 18-years-old and/or have a mild form of GD. In 1991, the Genzyme Corporation in cooperation with the NIH developed the first FDA-approved GD targeted ERT. With the introduction of freeze-dried analogue of human β-glucocerebrosidase (trade name CEREZYME®) and its ready-to-use formulation (Imiglucerase) in 1994, clinicians have been able to address the disease process itself, and therefore alleviate and even reverse many effects of Type 1 GD. CEREZYME® therapy is not a cure for GD; that is, it does not correct the underlying genetic defect. In order to continue to benefit from the treatment, symptomatic patients must receive intravenous infusions for the rest of their lives. The history and current perspective of CEREZYME® and Imiglucerase in the treatment of GD were recently reviewed (Deegan and Cox, 2012).

Taliglucerase alfa (trade name ELELYSO™) for injection is a plant-derived hydrolytic lysosomal glucocerebroside-specific enzyme indicated for long-term ERT for adults with a confirmed diagnosis of Type 1 GD. ELELYSO™ was approved by the United States Food and Drug Administration (FDA) on May 1, 2012, and is the first plant cell-based FDA-approved ERT indicated for the treatment of Type 1 GD. The benefits of taliglucerase alfa in the treatment of patients with GD were recently reviewed (Hollak, 2012).

Velaglucerase alfa (trade name VPRIV®) is a hydrolytic lysosomal glucocerebroside-specific enzyme indicated for long-term ERT for pediatric and adult patients with Type 1 GD. Velaglucerase alfa is derived from a human cell line and designed to have an amino acid sequence similar to the naturally occurring human β-glucocerebrosidase protein. It was approved for use by FDA on Feb. 26, 2010. The results from a randomized, double-blind, multinational, Phase 3 study of ERT with velaglucerase alfa in GD, were recently published (Gonzalez et al., 2013). The safety and efficacy results of velaglucerase alfa in this study have supported the approval of velaglucerase alfa in the United States and Europe and the emergence of a valuable treatment option for patients with Type 1 GD.

In addition to the aforementioned ERT drugs, Miglustat (OGT 918, N-butyl-deoxynojirimycin, ZAVESCA®) is a prescription substrate reduction therapy (SRT) medicine taken by mouth for adults with mild to moderate Type 1 GD. ZAVESCA® is used only in patients who cannot be treated with ERT. ZAVESCA® reduces the harmful buildup of glycosphingolipids (GSLs) throughout the body by reducing the amount of GSLs that the body produces. A retrospective analysis of miglustat for Type 1 GD has found that a combination therapy may offer GD patients better disease control (by employing more than one mechanism of action against the accumulation of glucosylceramide in cells), can be cost-effective by using reduced doses of both ERT and miglustat, and can provide an acceptable quality of life (Machaczka et al., 2012).

### Genome-Wide Association Study

Genotype-phenotype correlations are rather poor for GD and therefore predicting what subtype of GD a patient will develop is a major challenge (Goker-Alpan et al 2005); for example, siblings with the same genotype can present with widely-differing phenotypes (Eyal et al 1991, Amato et al 2004). Therefore, a role for modifier genes has been proposed as the underlying basis of phenotypic variation (Goker-Alpan et al 2005).

Modifier genes can be identified by at least two independent strategies:

i) Search for changes in genes that are involved in the development of specific symptoms. Using this strategy, SCARB2 was identified as a modifier gene of GD in a pair of siblings with discordant epileptic phenotypes (Velayati et al 2011); and

ii) Genome Wide Association Studies. Genome-wide association study (GWA study, or GWAS), also known as whole genome association study (WGA study, or WGAS), is an examination of many common genetic variants in different individuals to see if any variant is associated with a trait. GWAS typically focus on associations between single-nucleotide polymorphisms (SNPs) and traits like major diseases. GWAS normally compare the DNA of two groups of participants: people with the disease (cases) and similar people without (controls). Each person gives a sample of DNA, from which millions of genetic variants are read. If one type of the variant (one allele) is more frequent in people with the disease, the SNP is said to be “associated” with the disease. The associated SNPs are then considered to mark a region of the human genome which influences the risk of disease.

The GWAS approach was recently utilized in the study of Ashkenazi Jewish GD patients with Type 1 GD (GD1), homozygous for N370S mutation. The study revealed the candidacy of the CLN8 gene as a genetic modifier contributing to extreme phenotypic variation (Zhang, 2012).

### NMDA Receptors

The NMDA receptor is a hetero-tetramer between two obligatory GluN1 (also denoted as NR1) and two regionally localized GluN2 subunits (also denoted as NR2). Multiple receptor isoforms with distinct brain distributions and functional properties arise by selective splicing of the NR1 transcripts and differential expression of the NR2 subunits. While a single NR2 subunit is found in invertebrate organisms, four distinct isoforms of the NR2 subunit are expressed in vertebrates and are referred to with the nomenclature NR2A through D (coded by genes GRIN2A, GRIN2B, GRIN2C and GRIN2D, respectively). Whereas NR2B is predominant in the early postnatal brain, the number of NR2A subunits grows, and eventually the NR2A subunits outnumber NR2B. This is called NR2B-NR2A developmental switch, and is notable because of the different kinetics each NR2 subunit lends to the receptor (Liu et al., 2004). Several synonyms exist for the NR2B protein, such as NMDAR2B; glutamate [NMDA] receptor subunit epsilon-2; glutamate receptor subunit epsilon-2; glutamate receptor, ionotropic, N-methyl D-aspartate 2B; GRIN2B; hNR3; MGC142178; MGC142180; N-methyl D-aspartate receptor subtype 2B; N-methyl-D-aspartate receptor subunit 3; NMDE2; or NR3. The protein comprises 1484 amino acids, of which the first 26 amino acids are thought to function as a potential signal peptide.

### Glutamate and NMDA Receptors

Glutamate is the major excitatory amino acid neurotransmitter in the brain (Nicholls et al., 2012). Under physiological conditions, glutamate plays fundamental roles in brain communication and plasticity (Bliss and Collingridge 1993; Cooke and Bliss 2006) through activation of glutamate receptors (GluRs) (Traynelis et al., 2010). Excessive activation of GluRs during neurological injuries produces an excess of calcium influx into the cells mainly via a subtype of GluRs called NMDA receptors, leading to neuronal damage and eventual cell death, a process called excitotoxicity (Mehta et al., 2013). NMDA receptors can be divided into 2 groups, according to their distribution in neurons: synaptic and extra-synaptic. The synaptic receptors promote survival signaling cascades while the extra-synaptic mediate the toxic effects of excitotoxicity (Parsons and Raymond, 2014). Excitotoxicity due to excessive glutamate release occurs in several neurological conditions, such as in ischemic stroke (Kostandy, 2005), autism (Essa et al., 2013), amyotrophic lateral sclerosis (Costa et al., 2010), Parkinson's (Caudle and Zhang, 2009) and Alzheimer's disease (Kostandy, 2005).

### Memantine and NMDA Receptors

Memantine was the first in a novel class of Alzheimer's disease medications acting on the glutamatergic system by blocking NMDA receptors. It was first synthesized by Eli Lilly and Company in 1968. Memantine has been shown to have a modest effect in moderate-to-severe Alzheimer's disease and in dementia with Lewy bodies and is approved for these uses in humans. U.S. Pat. No. 5,061,703 describes the use of memantine (and other adamantine derivatives) in prevention of ischemic damage to the brain. U.S. Pat. No. 8,168,209 and U.S. Pat. No. 8,329,752 describe extended release oral dosage forms of memantine. On 2003, the U.S. Food and Drug Administration (FDA) approved memantine (under the trade name NAMENDA) for treatment of moderate to severe Alzheimer's type dementia.

In summary, ERT is currently administered intravenously for the non-neurological manifestations of GD Types 1 and 3, and no treatments are yet available for Type 2, the most fatal form of the disease, which presents in infancy and causes demise within the first two to three years.

There thus remains an unmet medical need in the field for modes of therapy that would delay, ameliorate or diminish neurological symptoms that manifest themselves in the neuropathic forms of GD.

### SUMMARY OF THE INVENTION

The present invention provides compositions and methods for treatment of neuropathic forms of Gaucher Disease (GD) comprising as an active ingredient at least one type of NMDA receptor antagonist. The present invention provides compositions and methods effective in ameliorating, at least partially, the neurological onset and/or progression of the disease. In particular the compositions of the present invention diminish or prevent neurological symptoms of the disease. According to some embodiments, the NMDA antagonist is a specific inhibitor of glutamatergic neurotransmission. According to some embodiments, the NMDA antagonist may be a less specific antagonist that acts on other neurotransmission pathways. According to some embodiments, the NMDA antagonist may have additional activities such as serotonergic, cholinergic or dopaminergic antagonist or agonist activities.

The present invention is based, at least in part, on models of GD in inbred mice that disclosed for the first time that polymorphisms in the gene Grin2b, encoding for subunit B of the NMDA glutamate receptor, is associated with GD progression. To discover novel genes involved in GD progression, GD was induced in inbred mice strains by injecting conduritol B epoxide (CBE), an irreversible glucocerebrosidase (GBA) inhibitor, and performing a Genome Wide Association Study (GWAS). The GWAS analysis identified Grin2b, the gene encoding for the subunit B of the NMDA glutamate receptor, as a potential modifier gene of GD progression.

Thus, the present invention provides, in one aspect, a pharmaceutical composition comprising at least one NMDA receptor antagonist or a pharmaceutically acceptable salt, hydrate or pharmaceutically active enantiomer thereof, for use in treating a neuropathic form of Gaucher Disease (GD).

According to an aspect of some embodiments of the present invention there is provided an NMDA receptor antagonist for use in treating a neuropathic form of Gaucher Disease (GD).

The present invention further provides, in another aspect, a method for treating a neuropathic form of GD in a patient in need thereof, comprising the step of administering a pharmaceutical composition comprising at least one NMDA receptor antagonist or a pharmaceutically acceptable salt, hydrate or pharmaceutically active enantiomer thereof to the patient, thereby treating the neuropathic form of GD.

According to an aspect of some embodiments of the present invention there is provided a method for treating a neuropathic form of Gaucher Disease (GD) in a patient in need thereof, comprising administering a therapeutically effective amount of an NMDA receptor antagonist to the patient, thereby treating the neuropathic form of GD.

The present invention also provides, in an aspect, the use of at least one NMDA receptor antagonist or a pharmaceutically acceptable salt, hydrate or pharmaceutically active enantiomer thereof in the manufacture of a medicament for treating a neuropathic form of GD.

According to an aspect of some embodiments of the present invention there is provided a use of at least one NMDA receptor antagonist in the manufacture of a medicament for treating a neuropathic form of Gaucher Disease (GD).

The present invention further provides, in another aspect, a kit comprising at least one container comprising a pharmaceutical composition, the pharmaceutical composition comprising at least one NMDA receptor antagonist or a pharmaceutically acceptable salt, hydrate or pharmaceutically active enantiomer thereof, for use in treating neuropathic forms of GD.

According to some embodiments of the invention, the NMDA receptor antagonist or the use comprises an agent selected from the group consisting of an enzyme replacement therapy agent, substrate reduction therapy agent and pharmacological chaperone therapy agent.

According to some embodiments of the invention, the method comprising administering to the patient a therapeutically effective amount of an agent selected from the group consisting of an enzyme replacement therapy agent, substrate reduction therapy agent and pharmacological chaperone therapy agent.

According to an aspect of some embodiments of the present invention there is provided a kit identified for use in treating neuropathic form of Gaucher Disease (GD), comprising a packaging material packaging an NMDA receptor antagonist and an agent selected from the group consisting of an enzyme replacement therapy agent, substrate reduction therapy agent and pharmacological chaperone therapy agent.

According to some embodiments of the invention, the NMDA receptor is an extra-synaptic NMDA receptor.

According to some embodiments of the invention, the NMDA receptor comprises a NR2B subunit.

In certain embodiments, the NMDA receptor antagonist preferentially binds to extra-synaptic NMDA receptors. In certain embodiments, the extra-synaptic NMDA receptors comprise a NR2B subunit.

In certain embodiments, the NMDA receptor antagonist binds to an NMDA receptor of a neuronal cell having β-glucocerebrosidase activity deficiency relative to corresponding healthy neuronal cells. In certain embodiments, the cell comprises a mutation in the GBA1 gene. In certain embodiments, the cell expresses a mutated β-glucocerebrosidase enzyme. In certain embodiments, the neuronal cell is a central nervous system (CNS) neuron.

In certain embodiments, the NMDA receptor antagonist is selected from the group consisting of memantine, nitromemantine, neramexane, ketamine, amantadine, dextromethorphan, L-687,384, amitriptyline, 1-benzyl-6′-methoxy-6′,7′-dihydrospiro[piperidine-4,4′-thieno[3.2-c]pyran], ifenprodil, orphenadrine, kynurenic acid, felbamate, D(−)-AP-5, (±)-CPP, EAA-090, TCN-201, AP-5, AZD6765, SDZ 220-581, (±)-norketamine, eliprodil, dextrorphan, 5,7-dichlorokynurenic acid monohydrate, [Glu3,4,7,10,14]-Conantokin G, D-AP-7, MD-Ada, AP-7, Ro 8-4304, spermine, Ro 25-6981, DCQX, traxoprodil, MDL 105,519, fanapanel, metaphit, Ro 25-6981, NAAG, 5-fluoroindole-2-carboxylic acid, (S)-(−)-4-oxo-2-azetidinecarboxylic acid, benzyl (S)-(−)-4-oxo-2-azetidinecarboxylate and (±)-α-amino-3-carbomethoxy-5-methylisoxazole-4-propanoic acid, and pharmaceutically acceptable salts, hydrates and pharmaceutically active enantiomers thereof. Each possibility represents a separate embodiment of the invention.

In certain embodiments, the NMDA receptor antagonist is selected from the group consisting of memantine, nitromemantine, neramexane, ketamine, amantadine, dextromethorphan, L-687,384, amitriptyline, 1-benzyl-6′-methoxy-6′,7′-dihydrospiro-[piperidine-4,4′-thieno[3.2-c]pyran], eliprodil, ifenprodil, orphenadrine, kynurenic acid, felbamate, and pharmaceutically acceptable salts, hydrates and pharmaceutically active enantiomers thereof. Each possibility represents a separate embodiment of the invention.

In certain embodiments, the NMDA receptor antagonist is selected from the group consisting of memantine, nitromemantine, neramexane, and pharmaceutically acceptable salts, hydrates and pharmaceutically active enantiomers thereof. Each possibility represents a separate embodiment of the invention.

According to some embodiments of the invention, the NMDA receptor antagonist is selected from the group consisting of memantine, eliprodil and ifenprodil or a pharmaceutically acceptable salt, hydrate or pharmaceutically active enantiomer thereof.

In certain embodiments, the NMDA receptor antagonist is memantine (3,5-dimethyl-1-adamantanamine hydrochloride) or a pharmaceutically acceptable salt, hydrate or pharmaceutically active enantiomer thereof.

In certain embodiments, the NMDA receptor antagonist also has serotonergic activity. In certain embodiments, the NMDA receptor antagonist is also a 5-HT₃ receptor antagonist.

In certain embodiments, the NMDA receptor antagonist also has cholinergic activity. In certain embodiments, the NMDA receptor antagonist is also a nicotinic acetylcholine receptor (nAChR) antagonist.

In certain embodiments, the NMDA receptor antagonist having nAChR antagonist activity is selected from the group consisting of amantadine and dextromethorphan, and pharmaceutically acceptable salts, hydrates and pharmaceutically active enantiomers thereof. Each possibility represents a separate embodiment of the invention.

In certain embodiments, the NMDA receptor antagonist also has dopaminergic activity. In certain embodiments, the NMDA receptor antagonist is also a dopamine D₂ receptor agonist.

In certain embodiments, the NMDA receptor antagonist also has neuro-protective activity. In certain embodiments, the NMDA receptor antagonist is also a sigma-1 receptor agonist.

In certain embodiments, the NMDA receptor antagonist having sigma-1 receptor agonist activity is selected from the group consisting of L-687,384, amitriptyline and 1-benzyl-6′-methoxy-6′,7′-dihydrospiro[piperidine-4,4′-thieno[3.2-c]pyran], and pharmaceutically acceptable salts, hydrates and pharmaceutically active enantiomers thereof. Each possibility represents a separate embodiment of the invention.

In certain embodiments, the NMDA receptor antagonist has neuro-protective activity.

In certain embodiments, the pharmaceutical composition described above is for use in treating sub-acute, chronic neuropathic GD Type 3.

According to some embodiments of the invention, the GD is sub-acute, chronic neuropathic GD Type 3.

In certain embodiments, the GD Type 3 is GD Type 3a.

In certain embodiments, the GD Type 3 is GD Type 3b.

According to some embodiments of the invention, the NMDA receptor antagonist is formulated for oral delivery.

In certain embodiments, the pharmaceutical composition described above is formulated for oral delivery. In certain embodiments the composition for oral delivery is formulated for sustained release.

According to some embodiments of the invention, the NMDA receptor antagonist is formulated for injection.

In certain embodiments, the pharmaceutical composition described above is formulated for injection.

According to an aspect of some embodiments of the present invention there is provided a method of diagnosing neuropathic form of Gaucher Disease (GD) in a patient, the method comprising determining in a biological sample of the patient a sequence variation that affects an amount of an expression product of a Grin2B gene and/or an amount of an expression product of a Grin2B gene, wherein presence of the sequence variation, an amount of the expression product above a predetermined level and/or increased amount of the expression product relative to a biological sample of a healthy patient or a patient diagnosed with GD type I is indicative of a neuropathic form of GD, thereby diagnosing neuropathic form of GD in the patient.

According to some embodiments of the invention, the sequence variation is in a non-coding sequence of the Grin2B gene.

According to some embodiments of the invention, the sequence variation is determined using DNA sequencing.

According to some embodiments of the invention, the amount of expression is determined using an RNA and/or a protein detection method.

According to some embodiments of the invention, the detection method is selected from the group consisting of PCR, oligonucleotide microarray, immunoprecipitation, Western blot analysis and FACS.

According to some embodiments of the invention, the level of expression is determined by hybridizing the biological sample or fractions or extracts thereof of the patient with an oligonucleotide which specifically hybridizes with a polynucleotide expressed from the Grin2B gene and/or by contacting the biological sample or fractions or extracts thereof of the patient with an antibody which specifically binds a polypeptide expressed from the Grin2B gene.

According to some embodiments of the invention, patient is diagnosed with GD.

According to an aspect of some embodiments of the present invention there is provided a composition of matter comprising a polynucleotide sample of a patient diagnosed with Gaucher Disease (GD); and an oligonucleotide capable of specifically hybridizing with a polynucleotide expressed from a Grin2B gene and optionally wherein the oligonucleotide is labeled.

According to an aspect of some embodiments of the present invention there is provided a composition of matter comprising a polypeptide sample of a patient diagnosed with Gaucher Disease (GD); and an antibody capable of specifically binding a polypeptide expressed from a Grin2B gene and optionally a secondary antibody.

According to some embodiments of the invention, there is provided a method of treating a patient diagnosed with neuropathic form of Gaucher Disease (GD), the method comprising:

(a) diagnosing the patient according to the method; and wherein presence of the sequence variation and/or an amount of the expression product above a predetermined level and/or increased amount expression product relative to a biological sample of a healthy patient or a patient diagnosed with GD type I is indicated,

(b) treating the patient with a NMDA receptor antagonist, thereby treating the patient diagnosed with neuropathic form of GD.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE SEVERAL VIEWS OF THE DRAWINGS

FIGS. 1A-C show that different mouse strains demonstrate different susceptibility to CBE treatment. FIG. 1A is a figure adapted from Kirby et al. Genetics. 2010 July; 185(3):1081-95 showing the phylogenetic tree of 94 mouse strains. The strains used in the instant application are highlighted in bold lines. FIG. 1B is a Kaplan-Meier survival curve illustrating the survival curves of 15 different CBE-treated mouse strains. Mice were treated daily with CBE (25 mg/kg) day starting at postnatal day (P) 8. FIG. 1C shows progression of motor coordination in representative mouse strains following PBS (control, black lines) or CBE (gray lines) administration, as assessed by the hanging wire test. Data is presented as mean±SEM (n=4-5), *P<0.05.

FIG. 2 is a pictogram illustrating that CBE-induced brain pathology is strain specific. AKR/J and BTBR T^{+Itpr3tf/J} mice were treated with CBE. Mice were sacrificed at P22 and P208, respectively, and the presence of CD68-positive cells in cortical layer V of the brain was analyzed. AKR/J mice developed a more rapid brain disease whereas BTBR T^{+Itpr3tf/J} mice are resistant.

FIGS. 3A-F shows cerebral GBA1 activity and lipidomic analysis. FIG. 3A is a bar graph demonstrating GBA1 activity in brains of PBS- (control, black bars) or CBE-treated (gray bars) mice harvested on P18. Data is presented as mean±SEM (n=2 by duplicates). FIG. 3B is a graph showing lack of correlation between GBA1 activity following CBE-treatment and mouse life span. FIGS. 3C-F are bar graphs demonstrating GlcCer levels (FIG. 3C), GlcSo levels (FIG. 3D), GalCer levels (FIG. 3E), and GalSo levels (FIG. 3F) in brains of PBS- (control, black bars) and CBE-treated (gray bars) strains evaluated on P18. Each panel has an insertion graph showing the lack of correlation between each lipid and mouse life span following CBE treatment. Data is presented as mean±SEM (n=2).

FIG. 4A is a Manhattan plot generated using EMMA for SNPs identified in 15 different mouse strains. FIG. 4B is a blow-out of the Manhattan plot of FIG. 3A, focusing on positions 130,000,000 to 140,000,000 on chromosome 6.

FIG. 5A show cortical mRNA levels of Grin2b as evaluated by qPCR in cortical homogenates derived from PBS- or CBE-treated mice on P18. The genotypes of the SNPs in rs29869040 which reside within the Grin2b gene are indicated. A/J and C57BL/6/JolaHsd (denoted as C57) represent the short living strains while NZW and FVB represent the long living strains. Results are expressed as mean±SEM (n=3). Cycle threshold (Ct) values were normalized to the levels of TATA box-binding protein (Tbp). *P<0.05 between the short and long lived strains, #P<0.05 between PBS and CBE-treated tissues.

FIG. 5B shows NR2B protein levels as evaluated by western blot analysis of frontal, occipital, parietal cortex and cerebellum of control, Type 1 GD, and Type 2 GD patients. GAPDH levels were used as loading controls.

FIG. 6 is a line graph illustrating how blocking NMDA receptor extends C3H mice survival. Male mice from the C3H/HeJ (C3H) strain were treated daily with CBE (25 mg/Kg day) (black line) or CBE (25 mg/Kg day) plus MK-801 (0.3 mg/Kg day) (gray line) starting at P8.

FIG. 7 show line graphs illustrating how activating NMDA receptor reduces mice survival. Male mice from BALB/cJ, FVB/NJ, 129S1/SvImJ, and BTBR T⁺ Itpr3^{tf/J} strains were treated daily with CBE (25 mg/kg day) (black lines) or CBE (25 mg/kg day) plus D-cycloserine (200 mg/kg day) (gray lines) starting at P8.

FIGS. 8A-C show line graphs illustrating how memantine, an NMDA receptor antagonist, extends the lifespan of neuropathic GD mice. FIG. 8A shows Kaplan-Meier survival curves of male mice from A/J (AJ), C3H/HeJ (C3H), DBA/2J (DBA), and C57BL6/JolaHsd (C57) strains treated daily with CBE (25 mg/kg day) (CBE, black lines) or CBE (25 mg/kg) plus memantine (3 mg/kg day) (CBE-M, gray lines) starting at P8. FIGS. 8B-C show Kaplan-Meier survival curves (FIG. 8B) and mean survival times (FIG. 8C) of A/J mice treated daily with CBE (50 mg/kg day) (n=5) or CBE (50 mg/kg day) plus memantine 3 mg/kg (denoted as 1X, n=3) or CBE plus memantine 30 mg/kg day (denoted as X10, n=4) starting at P8. Results in FIG. 8C are presented as mean±SEM. *P<0.05.

FIG. 9A shows progression of motor coordination in A/J PBS-treated mice (black continuous line) (n=5), A/J CBE-treated mice (gray dot) (n=4) and A/J CBE-memantine (dotted line) (n=5), as assessed by the hanging wire test. Results are mean±SEM. *P<0.05 compared to PBS controls.

FIG. 9B is a bar graph showing cerebral GBA1 activity following treatment with PBS, CBE or CBE plus memantine in A/J mouse brains harvested at P18 or P70. Data is presented as mean±SEM, n=2 biological replicates and 2 technical replicates *P<0.001.

FIGS. 10A-B show Kaplan-Meier survival curves (FIG. 10B) and mean survival times (FIG. 10B) of Gba^{flox/flox}; nestin-Cre mice treated daily with PBS (control, n=9), memantine 3 mg/kg (denoted as 1X, n=4) or memantine 30 mg/kg day (denoted as 10X, n=6) starting at P8. Results in FIG. 10B are presented as mean±SEM. *P<0.05.

FIG. 11 shows Kaplan-Meier survival curves of A/J mice treated daily with CBE (25 mg/kg day, black lines) or CBE (25 mg/kg day) plus Ifenprodil (gray line) starting at P8.

### DETAILED DESCRIPTION OF THE INVENTION

The invention is directed to means and methods for treating a subject diagnosed to be at risk of developing or afflicted with neuropathic forms of Gaucher Disease (GD). The method of the invention comprises administering to the subject a therapeutically effective amount of an NMDA receptor antagonist effective in inhibiting or blocking the activity of NMDA receptors. The invention is applicable to all neuropathic forms of GD, including Type 2, Type 3a and Type 3b.

The treatments available to date for lysosomal storage diseases resulting from an enzyme deficiency, including Type 1 GD, are highly expensive and not always effective. As for Type 2 and Type 3 GD, no efficient therapeutic approach for treating those conditions exists. Very little is known about the biochemical pathways that lead to pathological events inneuropathic (also known as neuronopathic) GD (nGD).

Clinically, GD is very heterogeneous and patients bearing identical mutations in the lysosomal glucocerebrosidase (GBA1) gene can present completely different manifestations, indicating the existence of modifier genes. The inventors of the present invention herein show, for the first time, that the Grin2b gene, encoding for the subunit B of the NMDA glutamate receptor, is associated with GD progression in mice afflicted with GD. In addition, the present invention now shows that mice demonstrating GD present extended life span when treated by NMDA receptor antagonists. Therefore, according to this invention sequence variations and mRNA and/or protein levels of NMDA can be used in nGD diagnosis; and compounds capable of inhibiting the activity of NMDA receptors are suitable for the treatment of nGD.

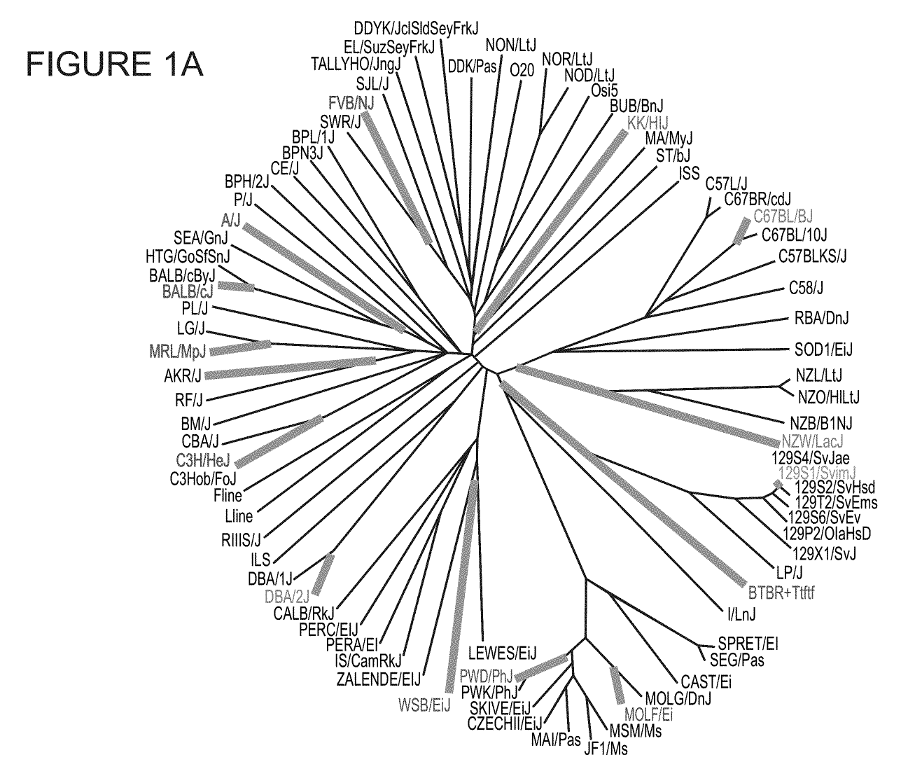

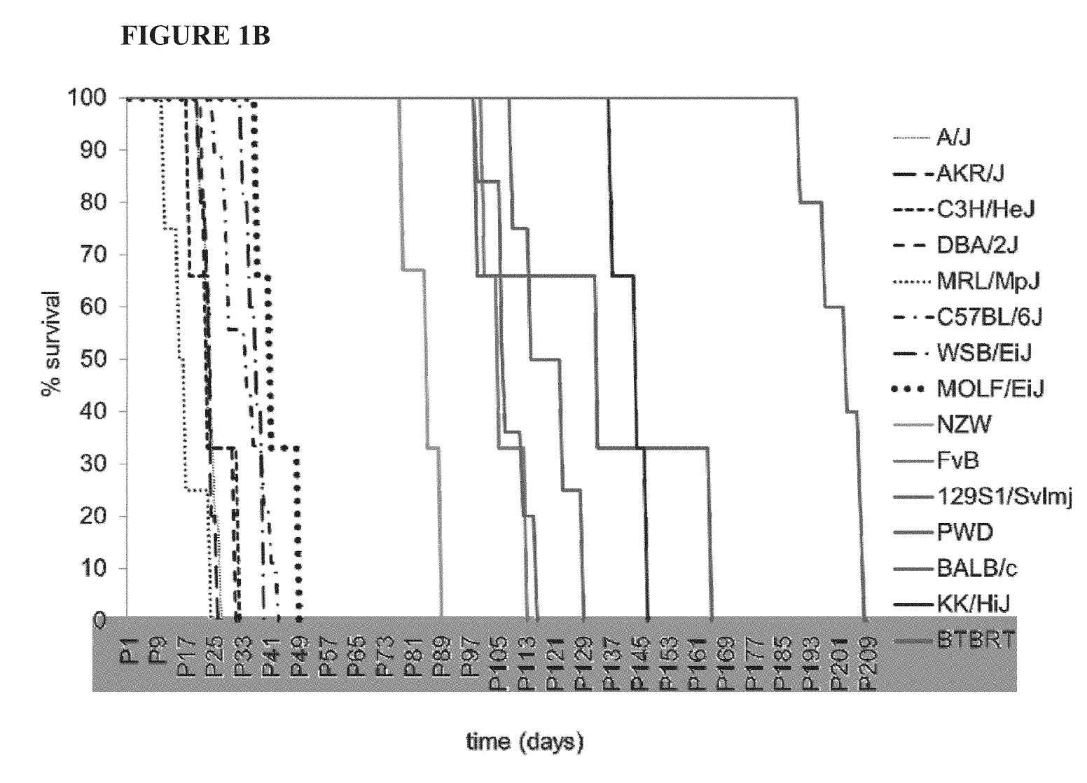

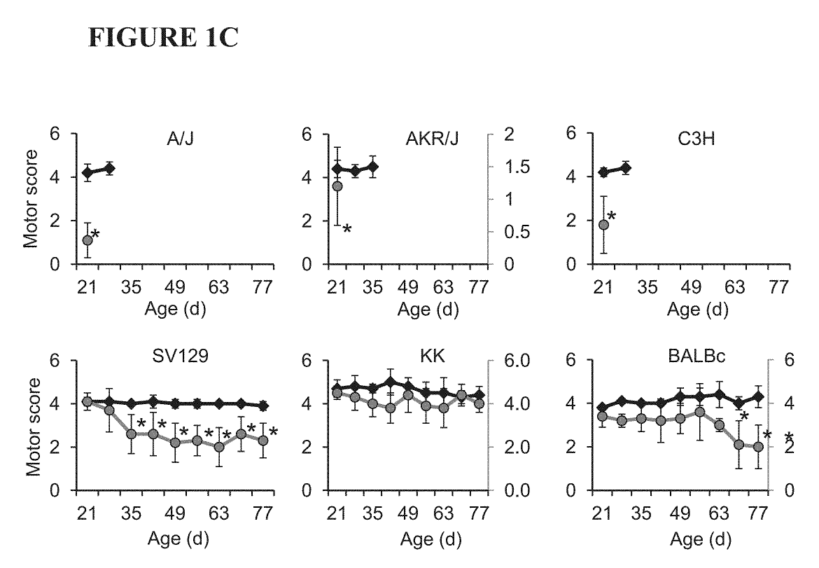

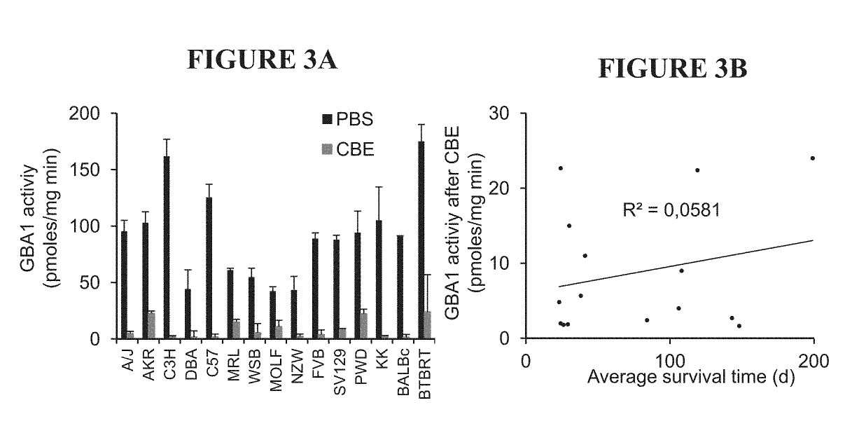

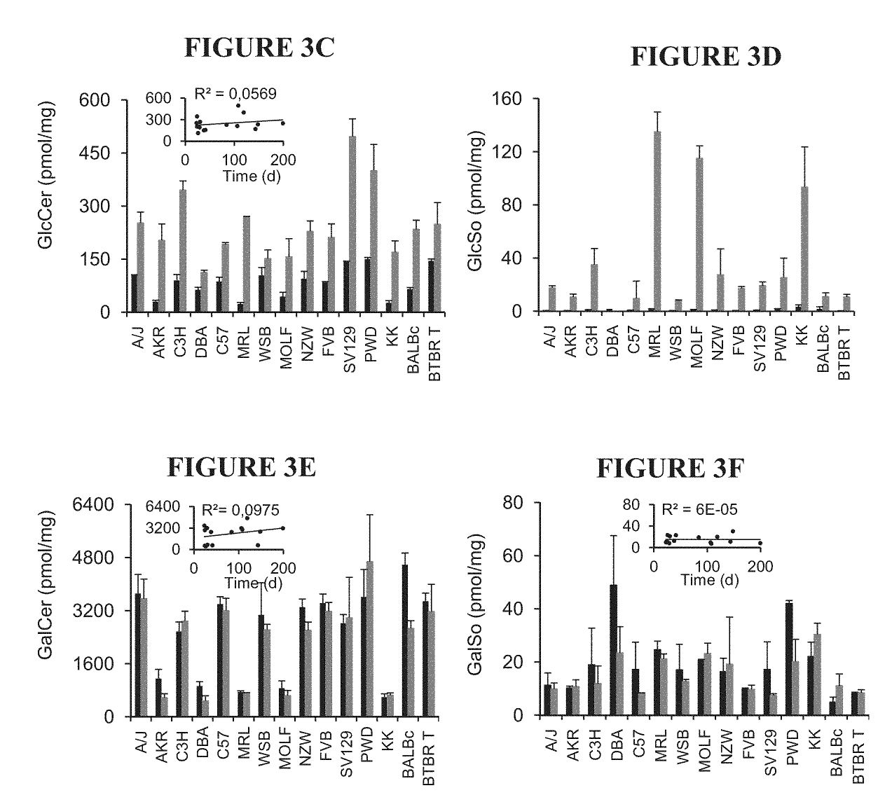

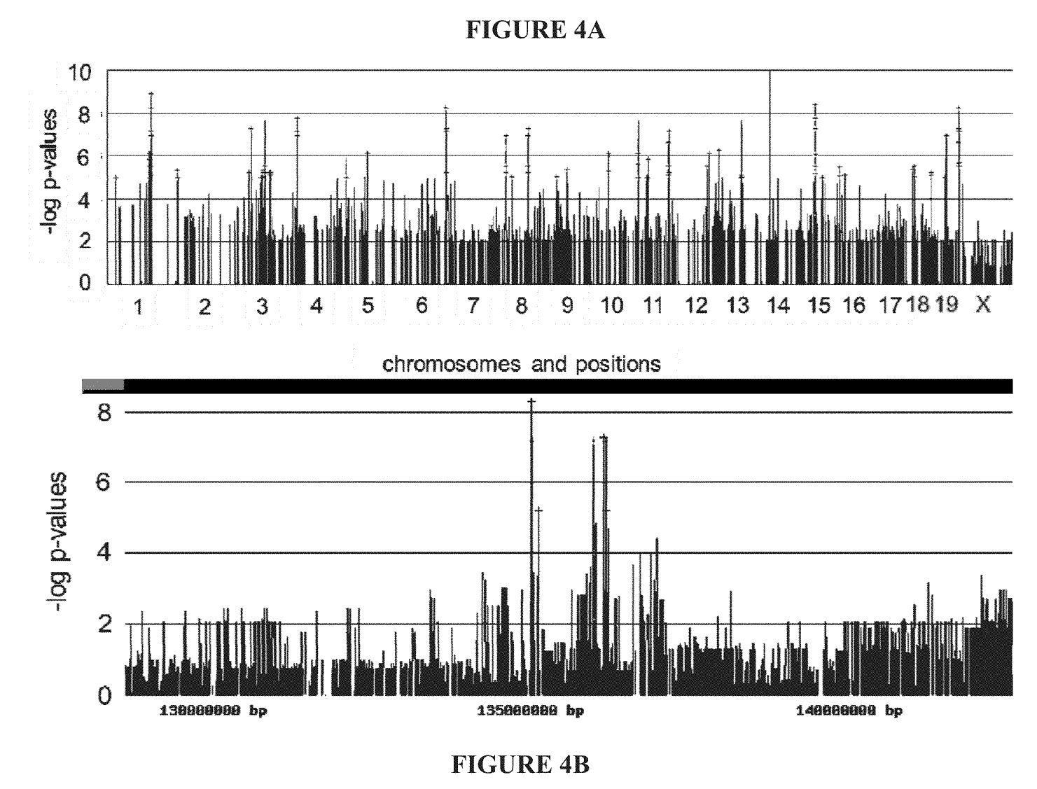

As is illustrated hereinunder and in the Examples section, which follows, the present inventors show that different mouse strains respond differently to GD induction by CBE, a response which is correlated to their genetic background. Furthermore, mouse strains that present short life span also present brain pathology and motor dysfunction and thus can be used as a mouse model for neuropathic Gaucher Disease (nGD) (Example 1, FIGS. 1A-C and 2 and Table 2). Importantly, there is no correlation between the survival rates of the mice and brain GBA1 activity nor GlcCer, GlcSo, GalCer and GalSo lipid levels (Example 2, FIGS. 3A-F), indicating the involvement of modifier genes in the nGD pathology. GWAS analysis has identified Grin2b, the gene encoding for the subunit B of the NMDA glutamate receptor, as a potential modifier gene of nGD progression (Example 4 FIGS. 4A-B and 5A-B). Specifically, the inventors have uncovered a single nucleotide variation (SNP ID rs29869040) in a non-coding sequence of the Grin2b gene which correlates to nGD.

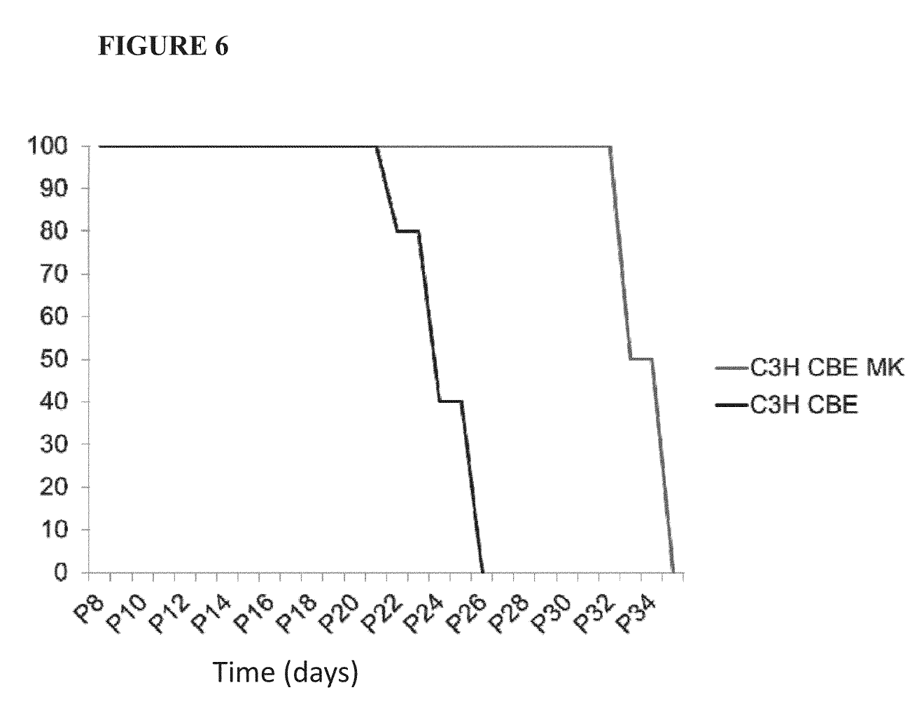

The present inventors further demonstrate that agonists of NMDA receptor (e.g. D-cycloserine) reduce survival of GD mice and antagonists of the NMDA receptor (e.g. MK801, Memantine and Ifenprodil) increase life expectancy and delay the progression of motor dysfunction in nGD mice while not affecting brain GBA1 inhibition (Examples 4-5, FIGS. 6-7, 8A-C, 9A-B, 10A-B and 11).

Consequently, the present teachings provide compositions comprising an NMDA receptor antagonist for use in treating neuropathic forms of Gaucher Disease (nGD) and methods of diagnosing nGD. Inspired by these surprising findings, the present invention provides, in one aspect, a pharmaceutical composition comprising at least one NMDA receptor antagonist or a pharmaceutically acceptable salt, hydrate or pharmaceutically active enantiomer thereof, for use in treating a neuropathic form of GD.

According to another aspect of the present invention, there is provided an NMDA receptor antagonist for use in treating a neuropathic form of GD.

Without wishing to be bound by any theory or mechanism of action, the NMDA receptor antagonist has neuro-protective activity. The term “neuro-protective activity” as used herein refers to the effects of reducing or ameliorating nervous insult, and protecting or reviving neuronal cells that have suffered nervous insult. As used herein, the term “nervous insult” refers to any damage to neuronal cell or tissue resulting from various causes such as metabolic, toxic, neurotoxic and chemical causes.

The present invention further provides, in another aspect, a method for treating a neuropathic form of GD in a patient in need thereof, comprising the step of administering a pharmaceutical composition comprising at least one NMDA receptor antagonist or a pharmaceutically acceptable salt, hydrate or pharmaceutically active enantiomer thereof to the patient, thereby treating the neuropathic form of GD.

According to another aspect of the present invention, there is provided a method for treating a neuropathic form of GD in a patient in need thereof, comprising administering a therapeutically effective amount of an NMDA receptor antagonist to the patient, thereby treating the neuropathic form of GD.

The present invention also provides, in an aspect, the use of at least one NMDA receptor antagonist or a pharmaceutically acceptable salt, hydrate or pharmaceutically active enantiomer thereof in the manufacture of a medicament for treating a neuropathic form of GD.

According to another aspect of the present invention, there is provided a use of at least one NMDA receptor antagonist in the manufacture of a medicament for treating a neuropathic form of GD.

The present invention further provides, in another aspect, a kit comprising at least one container comprising a pharmaceutical composition, the pharmaceutical composition comprising at least one NMDA receptor antagonist or a pharmaceutically acceptable salt, hydrate or pharmaceutically active enantiomer thereof, for use in treating neuropathic forms of GD.

The terms “treating” and “treatment” as used herein refer to abrogating, substantially inhibiting, slowing or reversing the progression of a condition, substantially delaying the appearance of clinical symptoms of a condition, substantially ameliorating clinical symptoms of a condition or substantially preventing the appearance of clinical symptoms of a condition. The term “treating” as used herein further refers to extending survival or delaying death of patients inflicted with a condition.

As used herein the phrases “patient in need thereof” and “subject in need thereof” which are interchangeably used herein, refer to a mammalian male or female subject (e.g., human being) who is diagnosed with the condition (i.e. neuropathic GD). In a specific embodiment, this term encompasses individuals who are at risk to develop the condition. The patient may be of any gender or at any age including neonatal, infant, juvenile, adolescent, adult and elderly adult. For diagnostic purposes according to some embodiments of the invention, the subject is further defined hereinbelow.

The terms “Gaucher's disease (GD)” or “Gaucher disease” as used herein, refers to a lysosomal storage disease (LSD) characterized by accumulation of glucosylceramide (GlcCer, also known as glucocerebroside) in cells, particularly in cells of the mononuclear cell lineage. Glucosylceramide can collect in the spleen, liver, kidneys, lungs, brain and bone marrow The disease is caused by a deficiency of the enzyme glucocerebrosidase (also known as beta-glucosidase, D-glucosyl-N-acylsphingosine glucohydrolase, GCD or GCase; EC 3.2.1.45), a lysosomal enzyme with glucosylceramidase activity that is needed to cleave, by hydrolysis, the beta-glucosidic linkage of glucosylceramide.

GD is divided into two major types: neuropathic and non-neuropathic disease, based on the particular symptoms of the disease. In non-neuropathic disease most organs and tissues can be involved, but not the brain. In neuropathic disease (nGD) the brain is also involved.

Type I (or non-neuropathic type, GD1) is the most common form of the disease, occurring in approximately 1 in 50,000 live births. It occurs most often among persons of Ashkenazi Jewish heritage. Symptoms may begin early in life or in adulthood and include enlarged liver and grossly enlarged spleen (known together as ‘hepatosplenomegaly’); the spleen can rupture and cause additional complications. Spleen enlargement and bone marrow replacement cause anemia, thrombocytopenia and leukopenia. Skeletal weakness and bone disease may be extensive. The brain is not affected pathologically, but there may be lung and, rarely, kidney impairment. Patients in this group usually bruise easily (due to low levels of platelets) and experience fatigue due to low numbers of red blood cells. Depending on disease onset and severity, type 1 patients may live well into adulthood. Some patients have a mild form of the disease or may not show any symptoms.

Neuropathic GD (nGD) as used herein encompasses both Type 2 and Type 3 GD.

GD type 2, also referred to as acute infantile neuropathic GD, typically begins within 6 months of birth and has an incidence rate around one 1 in 100,000 live births. Symptoms include an enlarged liver and spleen, extensive and progressive brain damage, eye movement disorders, spasticity, seizures, limb rigidity, and a poor ability to suck and swallow. Affected children usually die by age two.

According to specific embodiments, the neuropathic GD is GD Type 2.

GD type 3, also referred to as chronic neuropathic GD, can begin at any time in childhood or even in adulthood, and occurs in about one in 100,000 live births. It is characterized by slowly progressive, but milder neurologic symptoms compared to the acute or type 2 version. GD Type 3 has been divided into two variants, termed Types 3b and 3a. Type 3b has earlier onset of massive livers and spleens and the patients can also experience direct involvement of the lungs and rapidly progressive bony disease. Major symptoms include an enlarged spleen and/or liver, seizures, poor coordination, skeletal irregularities, eye movement disorders, blood disorders including anemia, and respiratory problems. Patients often live into their early teen years and adulthood.

According to specific embodiments, the neuropathic GD is the sub-acute, chronic neuropathic GD Type 3. In certain embodiments, the GD Type 3 is GD Type 3a. In certain embodiments, the GD Type 3 is GD Type 3b.

In certain embodiments, the NMDA receptor antagonist or the pharmaceutical composition comprising same is for use in treating at least one symptom related to the neuropathic forms of GD. In certain embodiments, the symptom is selected from the group consisting of convulsions, hypertonia, mental retardation, apnea, extensive and progressive brain damage, eye movement disorders, spasticity, seizures, limb rigidity, poor ability to suck and swallow, poor coordination, skeletal irregularities, blood disorders, anemia, muscle twitches (myoclonus), convulsions, dementia, ocular muscle apraxia, respiratory problems and any combination thereof. Each possibility represents a different embodiment of the invention.

As used herein, the term “NMDA receptor”, also known as N-methyl-D-aspartate receptor, refers to a glutamate receptor and ion channel protein which is a hetero-tetramer molecule that has at least one NR1 subunit (also known as GluN1) and at least one NR2 subunit (also known as GluN2). NMDA receptors can be divided into 2 groups, according to their distribution in neurons: synaptic and extra-synaptic. Typically, the synaptic receptors promote survival signaling cascades while the extra-synaptic mediate the toxic effects of excitotoxicity. According to specific embodiments, the NMDA receptor is an extra-synaptic NMDA receptor.

Multiple receptor isoforms with distinct brain distributions and functional properties arise by selective splicing of the NR1 transcripts and differential expression of the NR2 subunits. While a single NR2 subunit is found in invertebrate organisms, four distinct isoforms of the NR2 subunit are expressed in vertebrates and are referred to with the nomenclature NR2A through D (coded by genes GRIN2A, GRIN2B, GRIN2C and GRIN2D, respectively).

As detailed in the examples section below, it was found that Grin2b, the gene encoding for the subunit B of the NMDA glutamate receptor (NR2B), is a potential modifier gene of GD progression. Thus, in certain embodiments, the NMDA receptor, either synaptic or extra-synaptic, comprises a NR2B subunit. In particular embodiments, the extra-synaptic NMDA receptor comprises a NR2B subunit.

As used herein, the term “NR2B”, also known as NMDAR2B; Glutamate [NMDA] receptor subunit epsilon-2; glutamate receptor subunit epsilon-2; glutamate receptor, ionotropic, N-methyl D-aspartate 2B; GRIN2B; hNR3; MGC142178; MGC142180; N-methyl D-aspartate receptor subtype 2B; N-methyl-D-aspartate receptor subunit 3; NMDE2; or NR3, refers to refers to an expression product of the Grin2B gene. According to specific embodiments, the Grin2B gene refers to the human gene such as provided in the following Gene ID Number: 2904 (SEQ ID NO: 6). According to other specific embodiments the Grin2B gene refers to the mouse gene such as provided in the following Gene ID Number: 14812 (SEQ ID NO: 7). According to a specific embodiment, the NR2B protein refers to the human protein, such as provided in the following GenBank Number NP_000825 (SEQ ID NO: 8). According to a specific embodiment, the NR2B protein refers to the mouse protein, such as provided in the following GenBank Number NP_032197 (SEQ ID NO: 9).

The term “NMDA receptor antagonist” as used herein refers to a molecule that prevents and/or inhibits the biological function and/or expression of an N-methyl-D-aspartate receptor (NMDAR).

The antagonist may be a reversible or an irreversible antagonist.

The antagonist may be a competitive or a non-competitive antagonist.

According to specific embodiments the antagonist is a non-competitive antagonist.

According to other specific embodiments, the antagonist inhibits the biological function (e.g. ligand binding, opening of an ion channel) of the receptor e.g., as detected by e.g. electrophysiological recordings, calcium dyes, or calcium signaling. The reduction may be by at least a 10%, at least 20%, at least 30%, at least 40%, at least 50%, at least 60%, at least 70%, at least 80%, at least 90%, at least 95% or at least 99% as compared to same in the absence of the antagonist. According to specific embodiments, the antagonist completely prevents the biological function (e.g. ligand binding, opening of the ion channel) of the receptor.

According to specific embodiments, the antagonist directly binds the receptor, thereby preventing and/or inhibiting the activity and/or expression of the receptor. Binding assays are well known in the art and include e.g. BiaCore, high-performance liquid chromatography (HPLC) or flow cytometry.

According to other specific embodiments, the antagonist indirectly binds the NMDA receptor by acting through an intermediary molecule, for example the antagonist binds to or modulates a molecule that in turn binds to or modulates the NMDA receptor.

As described above, it is known that NMDA receptors can be divided into 2 groups, synaptic receptors, which promote survival signaling cascades; and extra-synaptic receptors, which mediate the toxic effects of excitotoxicity (Parsons and Raymond, 2014). It is thus speculated, without being limited to any theory or mechanism, that neuro-protection may be achieved by specifically targeting the excitotoxicity-promoting extra-synaptic receptors. Thus, in certain embodiments, the NMDA receptor antagonist preferentially binds to extra-synaptic NMDA receptors.

Thus, according to specific embodiments, the NMDA receptor antagonist prevents and/or inhibits excessive calcium influx via the NMDA receptor.

Certain NMDA receptor antagonists, such as MK-801, cannot be used to treat human patients due to their related toxicity, blocking not just the toxic effects of excessive calcium influx via extra-synaptic receptors, but also crucial normal functions of glutamate signaling.

Thus, according to specific embodiments, the NMDA receptor antagonist does not interfere with glutamate signaling via the NMDA receptor.

Furthermore, the use of NMDA receptor antagonists devoid of unacceptable side-effects such as hallucinogenic effects is warranted. Therefore, other NMDA receptor antagonists, such as memantine, which preferentially block extra-synaptic NMDA receptors, are favored, due to their specificity and safety.

The term “preferentially binds extra-synaptic NMDA receptors” as used herein refers to any NMDA receptor antagonist which binds extra-synaptic NMDA receptors without interfering with normal functions of glutamate signaling.

In certain embodiments, the NMDA receptor antagonist preferentially binds to a subset of NR2 subunits. In certain embodiments, the NMDA receptor antagonist preferentially binds to NR2B subunits. In certain embodiments, the NMDA receptor antagonist binds specifically to NR2B subunits without cross reactivity with other NR2 subunits.

In certain embodiments, the NMDA receptor antagonist binds to an NMDA receptor of a neuronal cell having β-glucocerebrosidase activity deficiency relative to corresponding healthy neuronal cells. The phrase “neuronal cell having β-glucocerebrosidase activity deficiency” as used herein further refers to a neuronal cell having significantly lower β-glucocerebrosidase activity than the β-glucocerebrosidase activity in normal, healthy neuronal cells.

Preventing and/or inhibiting the biological function of an NMDA receptor can be effected at the protein level but may also be effected at the genomic and/or the transcript level using a variety of molecules which interfere with transcription and/or translation of a receptor.

Therefore, non-limiting examples of antagonists that can be used according to some embodiments of the invention include small molecules, antibodies, inhibitory peptides, enzymes that cleave the polypeptide, aptamers homologous recombination agents, site specific endonucleases and RNA silencing agents.

Platform technologies for passing the blood brain barrier (BBB) are available and known in the art and include, but not limited to, directed liposomes, nanoparticles, microbubbles, phages by nasal administration, exosomes, pro-drugs and peptide-masking. Other exemplary approaches for drug delivery to the central nervous system (CNS) are further described hereinbelow.

Non limiting examples of agents that can function as antagonists are described in details hereinbelow.

Suppressing Biological Function at the Polypeptide Level

According to specific embodiments, the antagonistic agent is a molecule which interferes with the receptor function (e.g. catalytic or interaction) by binding to and/or cleaving the receptor. Such molecules can be, but are not limited to, small molecules, inhibitory peptides, enzymes that cleaves the receptor, adnectins, affibodies, avimers, anticalins, tetranectins, DARPins, and engineered Kunitz-type inhibitors wherein each possibility is a separate embodiment of the invention.

According to a specific embodiment, the antagonist is a small molecule.

According to a specific embodiment, the antagonist is a peptide molecule.

It will be appreciated that a non-functional analogue of at least a catalytic or binding portion of an inhibitory peptide can be also used as an antagonist.

Many compounds acting as NMDA receptor antagonists are known in the field, therefore the specific NMDA receptor antagonists specified below are by no means limiting to the scope of the invention.

Table 1 provides a list of several known NMDA receptor antagonists.

TABLE 1

| Additional | | NMDA receptor |
|---|---|---|
| activity | Formula | antagonist |
| stimulates | 3,5-Dimethyl-1-adamantanamine hydrochloride | Memantine |
| dopamine | | |
| release | | |
| selectively | 3-amino-5,7-diethyladamantan-1-yl nitrate | Nitromemantine |
| inhibits | | |
| extrasynaptic | | |
| over | | |
| physiological | | |
| synaptic | | |
| NMDA | | |
| receptor | | |
| activity | | |
| nAChR | 1,3,3,5,5-pentamethylcyclohexanamine | Neramexane |
| antagonist | | |
| D₂ receptor | (RS)-2-(2-Chlorophenyl)-2- | Ketamine |
| partial | (methylamino)cyclohexanone | |
| agonist | | |
| nAChR | adamantan-1-amine | Amantadine |
| antagonist | | |
| sigma-1 | (4bS,8aR,9S)-3-Methoxy-11-methyl-6,7,8,8a,9,10- | Dextromethorphan |
| receptor | hexahydro-5H-9,4b-(epiminoethano)phenanthrene | |
| agonist; | | |
| nAChR | | |
| antagonist | | |
| sigma-1 | 1′-benzylspiro[2,3-dihydro-1H-naphthalene-4,4′- | L-687,384 |
| receptor | piperidine] | |
| agonist | | |
| sigma-1 | 3-(10,11-dihydro-5H-dibenzo[a,d]cycloheptene-5- | Amitriptyline |
| receptor | ylidene)-N,N-dimethylpropan-1-amine | |
| agonist | | |
| sigma-1 | 1-benzyl-6′-methoxy-6′,7′-dihydrospiro[piperidine-4,4′- | |
| receptor | thieno[3.2-c]pyran] | |
| agonist | | |
| α-adrenergic | α-(4-Hydroxyphenyl)-β-methyl-4-benzyl-1- | Ifenprodil |
| central and | piperidineethanol (+)-tartrate salt | |
| peripheral | | |
| vasodilator; | | |
| α2 | | |
| adrenergic | | |
| receptor | | |
| ligand | | |
| Muscarinic | β-Dimethylaminoethyl 2-methylbenzhydryl ether citrate | Orphenadrine |
| receptor | salt | |
| antagonist; | | |
| H₁ histamine | | |
| receptor | | |
| antagonist; | | |
| reported to | | |
| inhibit the | | |
| noradrenergic | | |
| transporter | | |
| blocks | 4-Hydroxyquinoline-2-carboxylic acid | Kynurenic acid |
| nicotinic | | |
| acetycholine | | |
| receptors | | |
| has γ- | 2-Phenyl-1,3-propanediol dicarbamate | Felbamate |
| aminobutyric | | |
| acid | | |
| (GABA_{A}) | | |
| receptor | | |
| agonist | | |
| properties | | |
| | D(−)-2-Amino-5-phosphonopentanoic acid | D(−)-AP-5 |
| | (±)-3-(2-Carboxypiperazin-4-yl)propyl-1-phosphonic acid | (±)-CPP |
| | P-[2-(8,9-dioxo-2,6-diazabicyclo[5.2.0]non-1(7)-en-2- | EAA-090 |
| | yl)ethyl]-phosphonic acid | |
| | 3-Chloro-4-fluoro-N-[(4-{[2- | TCN-201 |
| | (phenylcarbonyl)hydrazino]carbonyl}phenyl)methyl]benzenesulfonamide | |
| | DL-2-Amino-5-phosphonovaleric acid | AP-5 |
| | (αS)-α-Phenyl-2-pyridineethanamine dihydrochloride | AZD6765 |
| | (S)-α-Amino-2-chloro-5-(phosphonomethyl)[1,1?- | SDZ 220-581 |
| | biphenyl]-3-propanoic acid hydrochloride | |
| | (±)-2-Amino-2-(2-chlorophenyl)cyclohexanone | (±)-Norketamine |
| | hydrochloride | |
| | α-(4-Chlorophenyl)-4-[(4-fluorophenyl)methyl]-1- | Eliprodil |
| | piperidineethanol | |
| | (+)-3-Hydroxy-N-methylmorphinan (+)-tartrate salt | Dextrorphan |
| | 5,7-Dichloro-4-hydroxyquinoline-2-carboxylic acid | 5,7- |
| | monohydrate | Dichlorokynurenic |
| | | acid |
| | | monohydrate |
| | Gly-Glu-Glu-Glu-Leu-Gln-Glu-Asn-Gln-Glu-Leu-Ile-Arg- | [Glu3,4,7,10,14]- |
| | Glu-Lys-Ser-Asn (SEQ ID NO: 1) | Conantokin G |
| | D(−)-2-Amino-7-phosphonoheptanoic acid | D-AP-7 |
| | 1-N-Methylamino-3,5-dimethyladamantane hydrochloride | MD-Ada |
| | DL-2-Amino-7-phosphonoheptanoic acid | AP-7 |
| | 4-{3-[4-(4-fluorophenyl)-3,6-dihydro-2H-pyridin-1-yl]-2- | Ro 8-4304 |
| | hydroxy-propoxy}-benzamide | |
| | N,N′-Bis(3-aminopropyl)-1,4-diaminobutane | Spermine |
| | [R-(R*,S*)]-α-(4-Hydroxyphenyl)-β-methyl-4- | Ro 25-6981 |
| | (phenylmethyl)-1-piperidinepropanol hydrochloride | |
| | hydrate | |
| | 6,7-Dichloro-2,3-quinoxalinedione | DCQX |
| | (1S,2S)-1-(4-hydroxy-phenyl)-2-(4-hydroxy-4- | Traxoprodil |
| | phenylpiperidino)-1-propanol | |
| | (E)-4,6-Dichloro-3-(2-phenyl-2-carboxyethenyl)indole-2- | MDL 105,519 |
| | carboxylic acid | |
| | [[3,4-Dihydro-7-(4-morpholinyl)-2,3-dioxo-6- | Fanapanel |
| | (trifluoromethyl)-1(2H)-quinoxalinyl]methyl]-phosphonic | |
| | acid hydrate | |
| | 1-[1-(3-Isothiocyanato)phenyl]cyclohexylpiperidine | Metaphit |
| | methanesulfonate salt | |
| | 1-Piperidinepropanol | Ro 25-6981 |
| | N-Acetylaspartylglutamic acid | NAAG |
| | 5-Fluoroindole-2-carboxylic acid | |
| | (S)-(−)-4-Oxo-2-azetidinecarboxylic acid | |
| | Benzyl (S)-(−)-4-oxo-2-azetidinecarboxylate | |
| | (±)-α-Amino-3-carbomethoxy-5-methylisoxazole-4- | |
| | propanoic acid | |

In certain embodiments, the at least one NMDA receptor antagonist is selected from the group consisting of memantine, nitromemantine, neramexane, ketamine, amantadine, dextromethorphan, L-687,384, amitriptyline, 1-benzyl-6′-methoxy-6′,7′-dihydrospiro[piperidine-4,4′-thieno[3.2-c]pyran], ifenprodil, orphenadrine, kynurenic acid, felbamate, D(−)-AP-5, (±)-CPP, EAA-090, TCN-201, AP-5, AZD6765, SDZ 220-581, (±)-norketamine, eliprodil, dextrorphan, 5,7-dichlorokynurenic acid monohydrate, [Glu3,4,7,10,14]-Conantokin G, D-AP-7, MD-Ada, AP-7, Ro 8-4304, spermine, Ro 25-6981, DCQX, traxoprodil, MDL 105,519, fanapanel, metaphit, Ro 25-6981, NAAG, 5-fluoroindole-2-carboxylic acid, (S)-(−)-4-oxo-2-azetidinecarboxylic acid, benzyl (S)-(−)-4-oxo-2-azetidinecarboxylate and (±)-α-Amino-3-carbomethoxy-5-methylisoxazole-4-propanoic acid, and pharmaceutically acceptable salts, hydrates and pharmaceutically active enantiomers thereof. Each possibility represents a separate embodiment of the invention.

In certain embodiments, at least one NMDA receptor antagonist is selected from the group consisting of memantine, nitromemantine, neramexane, ketamine, amantadine, dextromethorphan, L-687,384, amitriptyline, 1-benzyl-6′-methoxy-6′,7′-dihydrospiro[piperidine-4,4′-thieno[3.2-c]pyran], ifenprodil, eliprodil, orphenadrine, kynurenic acid, felbamate, and pharmaceutically acceptable salts, hydrates and pharmaceutically active enantiomers thereof. Each possibility represents a separate embodiment of the invention.

In certain embodiments, at least one NMDA receptor antagonist is selected from the group consisting of memantine, nitromemantine, neramexane, and pharmaceutically acceptable salts, hydrates and pharmaceutically active enantiomers thereof. Each possibility represents a separate embodiment of the invention.

According to specific embodiments, the NMDA receptor antagonist is selected from the group consisting of memantine, eliprodil and ifenprodil or a pharmaceutically acceptable salt, hydrate or pharmaceutically active enantiomer thereof.

In certain embodiments, the NMDA receptor antagonist is memantine (3,5-dimethyl-1-adamantanamine hydrochloride) or a pharmaceutically acceptable salt, hydrate or pharmaceutically active enantiomer thereof. Each possibility represents a separate embodiment of the invention.

According to specific embodiments, the antagonistic agent is an antibody. According to specific embodiments, the antagonistic antibody specifically binds at least one epitope of an NMDA receptor. According to specific embodiments, the antibody can cross the BBB.

As used herein, the term “epitope” refers to any antigenic determinant on an antigen to which the paratope of an antibody binds. Epitopic determinants usually consist of chemically active surface groupings of molecules such as amino acids or carbohydrate side chains and usually have specific three dimensional structural characteristics, as well as specific charge characteristics.

The term “antibody” as used in this invention includes intact molecules as well as functional fragments thereof, such as Fab, F(ab′)2, Fv, scFv, dsFv, or single domain molecules such as VH and VL that are capable of binding to an epitope of an antigen. The antibody may be mono-specific (capable of recognizing one epitope or protein), bi-specific (capable of binding two epitopes or proteins) or multi-specific (capable of recognizing multiple epitopes or proteins).

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDR HI or HI; CDR H2 or H2; and CDR H3 or H3) and three in each of the VL (CDR LI or LI; CDR L2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 1992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 1989.), a compromise between Kabat and Chothia using Oxford Molecular's AbM antibody modeling software (now Accelrys®, see, Martin et al., 1989, Proc. Natl Acad Sci USA. 86:9268; and world wide web site www.bioinf-org.uk/abs), available complex crystal structures as defined by the contact definition (see MacCallum et al., J. Mol. Biol. 262:732-745, 1996), the “conformational definition” (see, e.g., Makabe et al., Journal of Biological Chemistry, 283:1156-1166, 2008) and IMGT [Lefranc M P, et al. (2003) IMGT unique numbering for immunoglobulin and T cell receptor variable domains and Ig superfamily V-like domains. Dev Comp Immunol 27: 55-77].

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:

(i) Fv, defined as a genetically engineered fragment consisting of the variable region of the light chain (VL) and the variable region of the heavy chain (VH) expressed as two chains;

(ii) single chain Fv (“scFv”), a genetically engineered single chain molecule including the variable region of the light chain and the variable region of the heavy chain, linked by a suitable polypeptide linker as a genetically fused single chain molecule.

(iii) disulfide-stabilized Fv (“dsFv”), a genetically engineered antibody including the variable region of the light chain and the variable region of the heavy chain, linked by a genetically engineered disulfide bond.

(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH1 domains thereof;

(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);

(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and

(vii) Single domain antibodies or nanobodies are composed of a single VH or VL domains which exhibit sufficient affinity to the antigen.

The antibody may be monoclonal or polyclonal.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

It will be appreciated that for human therapy or diagnostics, humanized antibodies are preferably used. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

Once antibodies are obtained, they may be tested for activity, for example via ELISA.

Another agent which can be used as antagonist with some embodiments of the invention is an aptamer. As used herein, the term “aptamer” refers to double stranded or single stranded RNA molecule that binds to specific molecular target, such as a protein. Various methods are known in the art which can be used to design protein specific aptamers. The skilled artisan can employ SELEX (Systematic Evolution of Ligands by Exponential Enrichment) for efficient selection as described in Stoltenburg R, Reinemann C, and Strehlitz B (Biomolecular engineering (2007) 24(4):381-403).

Suppressing Biological Function at the Nucleic Acid Level

Down-regulation at the nucleic acid level is typically effected using a nucleic acid agent, having a nucleic acid backbone, DNA, RNA, mimetics thereof or a combination of same. The nucleic acid agent may be encoded from a DNA molecule or provided to the cell per se.

Thus, the antagonist of some embodiments of the invention can be an RNA silencing agent. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene. RNA silencing has been observed in many types of organisms, including plants, animals, and fungi.

As used herein, the term “RNA silencing agent” refers to an RNA which is capable of specifically inhibiting or “silencing” the expression of a target gene. In certain embodiments, the RNA silencing agent is capable of preventing complete processing (e.g, the full translation and/or expression) of an mRNA molecule through a post-transcriptional silencing mechanism. RNA silencing agents include non-coding RNA molecules, for example RNA duplexes comprising paired strands, as well as precursor RNAs from which such small non-coding RNAs can be generated. Exemplary RNA silencing agents include dsRNAs such as siRNAs, miRNAs and shRNAs.

In one embodiment, the RNA silencing agent is capable of inducing RNA interference.

In another embodiment, the RNA silencing agent is capable of mediating translational repression.

According to an embodiment of the invention, the RNA silencing agent is specific to the target RNA (e.g. Grin2b) and does not cross inhibit or silence other targets or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene; as determined by PCR, Western blot, Immunohistochemistry and/or flow cytometry.

RNA interference refers to the process of sequence-specific post-transcriptional gene silencing in animals mediated by short interfering RNAs (siRNAs).

Following is a detailed description on RNA silencing agents that can be used according to specific embodiments of the present invention.

DsRNA, siRNA and shRNA—The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

Accordingly, some embodiments of the invention contemplate use of dsRNA to downregulate protein expression from mRNA.

According to one embodiment dsRNA longer than 30 bp are used. Various studies demonstrate that long dsRNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-134].

According to some embodiments of the invention, dsRNA is provided in cells where the interferon pathway is not activated, see for example Billy et al., PNAS 2001, Vol 98, pages 14428-14433. and Diallo et al, Oligonucleotides, Oct. 1, 2003, 13(5): 381-392. doi:10.1089/154545703322617069.

According to an embodiment of the invention, the long dsRNA are specifically designed not to induce the interferon and PKR pathways for down-regulating gene expression. For example, Shinagwa and Ishii [Genes & Dev. 17 (11): 1340-1345, 2003] have developed a vector, named pDECAP, to express long double-strand RNA from an RNA polymerase II (Pol II) promoter. Because the transcripts from pDECAP lack both the 5′-cap structure and the 3′-poly(A) tail that facilitate ds-RNA export to the cytoplasm, long ds-RNA from pDECAP does not induce the interferon response.

Another method of evading the interferon and PKR pathways in mammalian systems is by introduction of small inhibitory RNAs (siRNAs) either via transfection or endogenous expression.

The term “siRNA” refers to small inhibitory RNA duplexes (generally between 18-30 base pairs) that induce the RNA interference (RNAi) pathway. Typically, siRNAs are chemically synthesized as 21 mers with a central 19 bp duplex region and symmetric 2-base 3′-overhangs on the termini, although it has been recently described that chemically synthesized RNA duplexes of 25-30 base length can have as much as a 100-fold increase in potency compared with 21 mers at the same location. The observed increased potency obtained using longer RNAs in triggering RNAi is suggested to result from providing Dicer with a substrate (27 mer) instead of a product (21 mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned, the RNA silencing agent of some embodiments of the invention may also be a short hairpin RNA (shRNA).

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. Some of the nucleotides in the loop can be involved in base-pair interactions with other nucleotides in the loop. Examples of oligonucleotide sequences that can be used to form the loop include 5′-CAAGAGA-3′ and 5′-UUACAA-3′ (International Patent Application Nos. WO2013126963 and WO2014107763). It will be recognized by one of skill in the art that the resulting single chain oligonucleotide forms a stem-loop or hairpin structure comprising a double-stranded region capable of interacting with the RNAi machinery.

Synthesis of RNA silencing agents suitable for use with some embodiments of the invention can be effected as follows. First, the receptor or the receptor subunit (e.g. Grin2B) mRNA sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl ChemBiochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (www.ambion.com/techlib/tn/91/912.html).

Second, potential target sites are compared to an appropriate genomic database (e.g., human, mouse, rat etc.) using any sequence alignment software, such as the BLAST software available from the NCBI server (www.ncbi.nlm.nih.gov/BLAST/). Putative target sites which exhibit significant homology to other coding sequences are filtered out.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA preferably include the same nucleotide composition as the siRNAs but lack significant homology to the genome. Thus, a scrambled nucleotide sequence of the siRNA is preferably used, provided it does not display any significant homology to any other gene.

For example, suitable siRNAs directed against Grin2B can be obtained from e.g. Sigma.

It will be appreciated that, and as mentioned hereinabove, the RNA silencing agent of some embodiments of the invention need not be limited to those molecules containing only RNA, but further encompasses chemically-modified nucleotides and non-nucleotides.

According to another embodiment the RNA silencing agent may be a miRNA.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor known as the pri-miRNA. The pri-miRNA may form a hairpin with a stem and loop.

The hairpin structure of the pri-miRNA is recognized by Drosha, which is an RNase III endonuclease. Drosha typically recognizes terminal loops in the pri-miRNA and cleaves the pri-miRNA with a staggered cut typical of RNase III endonucleases yielding a pre-miRNA stem loop with a 5′ phosphate and ˜2 nucleotide 3′ overhang. The pre-miRNA is then actively transported from the nucleus to the cytoplasm by Ran-GTP and the export receptor Ex-portin-5.

The double-stranded stem or the 5′ phosphate and 3′ overhang at the base of the stem loop of the pre-miRNA is then recognized by Dicer, which is also an RNase III endonuclease. Dicer then cleaves off the terminal loop two helical turns away from the base of the stem loop leaving an additional 5′ phosphate and ˜2 nucleotide 3′ overhang. The resulting siRNA-like duplex, which may comprise mismatches, comprises the mature miRNA and a similar-sized fragment known as the miRNA*. miRNA* sequences may be found in libraries of cloned miRNAs but typically at lower frequency than the miRNAs.

Although initially present as a double-stranded species with miRNA*, the miRNA eventually becomes incorporated as a single-stranded RNA into a ribonucleoprotein complex known as the RNA-induced silencing complex (RISC) while the miRNA* is removed and degraded.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al. (2005, Nat Genet 37-495).

The target sites in the mRNA may be in the 5′ UTR, the 3′ UTR or in the coding region.

miRNAs may direct the RISC to downregulate gene expression by either of two mechanisms: mRNA cleavage or translational repression. The miRNA may specify cleavage of the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA.

It will be appreciated from the description provided herein above that contacting cells with a miRNA may be effected by transfecting/loading the cells with e.g. the mature double stranded miRNA, the pre-miRNA or the pri-miRNA.

The pre-miRNA sequence may comprise from 45-90, 60-80 or 60-70 nucleotides.

The pri-miRNA sequence may comprise from 45-30,000, 50-25,000, 100-20,000, 1,000-1,500 or 80-100 nucleotides.

Antisense—Antisense is a single stranded RNA designed to prevent or inhibit expression of a gene by specifically hybridizing to its mRNA. Downregulation of a receptor can be effected using an antisense polynucleotide capable of specifically hybridizing with an mRNA transcript encoding the receptor or the receptor subunit (e.g. Grin2b).

Design of antisense molecules which can be used to efficiently downregulate a receptor must be effected while considering two aspects important to the antisense approach. The first aspect is delivery of the oligonucleotide into the cytoplasm of the appropriate cells, while the second aspect is design of an oligonucleotide which specifically binds the designated mRNA within cells in a way which inhibits translation thereof.

The prior art teaches of a number of delivery strategies which can be used to efficiently deliver oligonucleotides into a wide variety of cell types [see, for example, Jääskeläinen et al. Cell Mol Biol Lett. (2002) 7(2):236-7; Gait, Cell Mol Life Sci. (2003) 60(5):844-53; Martino et al. J Biomed Biotechnol. (2009) 2009:410260; Grijalvo et al. Expert Opin Ther Pat. (2014) 24(7):801-19; Falzarano et al, Nucleic Acid Ther. (2014) 24(1):87-100; Shilakari et al. Biomed Res Int. (2014) 2014: 526391; Prakash et al. Nucleic Acids Res. (2014) 42(13):8796-807 and Asseline et al. J Gene Med. (2014) 16(7-8):157-65].

In addition, algorithms for identifying those sequences with the highest predicted binding affinity for their target mRNA based on a thermodynamic cycle that accounts for the energetics of structural alterations in both the target mRNA and the oligonucleotide are also available [see, for example, Walton et al. Biotechnol Bioeng 65: 1-9 (1999)]. Such algorithms have been successfully used to implement an antisense approach in cells.

In addition, several approaches for designing and predicting efficiency of specific oligonucleotides using an in vitro system were also published (Matveeva et al., Nature Biotechnology 16: 1374-1375 (1998)].

Thus, the generation of highly accurate antisense design algorithms and a wide variety of oligonucleotide delivery systems, enable an ordinarily skilled artisan to design and implement antisense approaches suitable for downregulating expression of known sequences without having to resort to undue trial and error experimentation.

Nucleic acid agents can also operate at the DNA level as summarized infra.

Suppressing the biological function of a receptor can also be achieved by inactivating the gene (e.g., Grin2b) via introducing targeted mutations involving loss-of function alterations (e.g. point mutations, deletions and insertions) in the gene structure.

As used herein, the phrase “loss-of-function alterations” refers to any mutation in the DNA sequence of a gene which results in downregulation of the expression level and/or activity of the expressed product, i.e., the mRNA transcript and/or the translated protein. Non-limiting examples of such loss-of-function alterations include a missense mutation, i.e., a mutation which changes an amino acid residue in the protein with another amino acid residue and thereby abolishes the enzymatic activity of the protein; a nonsense mutation, i.e., a mutation which introduces a stop codon in a protein, e.g., an early stop codon which results in a shorter protein devoid of the enzymatic activity; a frame-shift mutation, i.e., a mutation, usually, deletion or insertion of nucleic acid(s) which changes the reading frame of the protein, and may result in an early termination by introducing a stop codon into a reading frame (e.g., a truncated protein, devoid of the enzymatic activity), or in a longer amino acid sequence (e.g., a readthrough protein) which affects the secondary or tertiary structure of the protein and results in a non-functional protein, devoid of the enzymatic activity of the non-mutated polypeptide; a readthrough mutation due to a frame-shift mutation or a modified stop codon mutation (i.e., when the stop codon is mutated into an amino acid codon), with an abolished enzymatic activity; a promoter mutation, i.e., a mutation in a promoter sequence, usually 5′ to the transcription start site of a gene, which results in down-regulation of a specific gene product; a regulatory mutation, i.e., a mutation in a region upstream or downstream, or within a gene, which affects the expression of the gene product; a deletion mutation, i.e., a mutation which deletes coding nucleic acids in a gene sequence and which may result in a frame-shift mutation or an in-frame mutation (within the coding sequence, deletion of one or more amino acid codons); an insertion mutation, i.e., a mutation which inserts coding or non-coding nucleic acids into a gene sequence, and which may result in a frame-shift mutation or an in-frame insertion of one or more amino acid codons; an inversion, i.e., a mutation which results in an inverted coding or non-coding sequence; a splice mutation i.e., a mutation which results in abnormal splicing or poor splicing; and a duplication mutation, i.e., a mutation which results in a duplicated coding or non-coding sequence, which can be in-frame or can cause a frame-shift.

According to specific embodiments los-of-function alteration of a gene may comprise at least one allele of the gene.

The term “allele” as used herein, refers to any of one or more alternative forms of a gene locus, all of which alleles relate to a trait or characteristic. In a diploid cell or organism, the two alleles of a given gene occupy corresponding loci on a pair of homologous chromosomes.

According to other specific embodiments loss-of-function alteration of a gene comprises both alleles of the gene.

Methods of introducing nucleic acid alterations to a gene of interest are well known in the art [see for example Menke D. Genesis (2013) 51:-618; Capecchi, Science (1989) 244:1288-1292; Santiago et al. Proc Natl Acad Sci USA (2008) 105:5809-5814; International Patent Application Nos. WO 2014085593, WO 2009071334 and WO 2011146121; U.S. Pat. Nos. 8,771,945, 8,586,526, 6,774,279 and UP Patent Application Publication Nos. 20030232410, 20050026157, US20060014264; the contents of which are incorporated by reference in their entireties] and include targeted homologous recombination (e.g. “Hit and run”, “double-replacement”), site specific recombinases (e.g. the Cre recombinase and the Flp recombinase), PB transposases (e.g. Sleeping Beauty, piggyBac, Tol2 or Frog Prince), genome editing by engineered nucleases (e.g. meganucleases, Zinc finger nucleases (ZFNs), transcription-activator like effector nucleases (TALENs) and CRISPR/Cas system) and genome editing using recombinant adeno-associated virus (rAAV) platform. Agents for introducing nucleic acid alterations to a gene of interest can be designed publically available sources or obtained commercially from Transposagen, Addgene and Sangamo Biosciences.

Methods for qualifying efficacy and detecting sequence alteration are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis.

Sequence alterations in a specific gene can also be determined at the protein level using e.g. chromatography, electrophoretic methods, immunodetection assays such as ELISA and western blot analysis and immunohistochemistry.

One of the primary manifestations of GD is the pathologic accumulation of large quantities of glucosylceramide (GlcCer) in neuronal cells. Thus, in certain embodiments, the β-glucocerebrosidase activity deficiency manifests in an elevated and/or pathological accumulation of GlcCer in neurons.

One of the key criteria distinguishing GD Type 2 and 3 from GD Type 1 is the involvement of the central nervous system (CNS) and appearance of CNS-related symptoms. Therefore, in certain embodiments, the neuronal cell is a central nervous system (CNS) neuron.

As it is now known that even the GBA1 mutation found in a carrier is a high risk factor for Parkinson's Disease, it is contemplated that treatment with the NMDA antagonists according to the invention may be beneficial even for such patients having the GBA1 mutation who are predisposed to PD. Without wishing to be bound by any theory or mechanism of action it is contemplated that such treatment might delay onset of disease.

According to specific embodiments, the NMDA receptor antagonist binds specifically the NMDA receptor with no cross reactivity with other receptors.

Many compounds acting as NMDA receptor antagonists are known to possess further biological activities (as shown for example in Table 1 above). Thus, according to other specific embodiments, the NMDA receptor antagonist can modulate the activity of other receptors.

As used herein, the term “modulate” refers to altering activity either by inhibiting (i.e. antagonist) or by activating (i.e. agonist) activity and/or expression of a receptor.

According to specific embodiments, modulates activity and/or expression is inhibits activity and/or expression.

According to specific embodiments, modulates activity and/or expression is activates activity and/or expression.

Thus, in certain embodiments, the NMDA receptor antagonist also has serotonergic activity. The term “serotonergic activity” as used herein refers to the ability of the NMDA receptor antagonist to also modulate the activity of a receptor of the 5-HT receptor family.

As used herein, the term “5-HT receptor family” refers to a group of proteins that function as receptors for serotonin. The group contains subset of proteins which are encoded by genes which exhibit homology of greater than 72% or higher with each other in their deduced amino acid sequences within presumed transmembrane regions (linearly contiguous stretches of hydrophobic amino acids, bordered by charged or polar amino acids, that are long enough to form secondary protein structures that span a lipid bilayer). Four human 5-HT receptor subfamilies are known to date: 5-HT₁, 5-HT₂, 5-HT₃, and 5-HT₄.

In certain embodiments, the NMDA receptor antagonist is also a 5-HT₃ receptor antagonist.

In certain embodiments, the NMDA receptor antagonist having 5-HT₃ receptor antagonist activity is ketamine, or pharmaceutically acceptable salts, hydrates or pharmaceutically active enantiomers thereof. Each possibility represents a separate embodiment of the invention.

In certain embodiments, the NMDA receptor antagonist also has cholinergic activity. The term “cholinergic activity” as used herein refers to the ability of the NMDA receptor antagonist to also modulate the activity of a receptor of the nicotinic acetylcholine receptor (nAChR) family. In certain embodiments, the NMDA receptor antagonist is also a nicotinic acetylcholine receptor (nAChR) antagonist.

As used herein, the term “nicotinic acetylcholine receptor (nAChR) family” refers to a group of proteins that function as receptors for acetylcholine that signal for muscular contractions upon a ligand binding. In certain embodiments, the NMDA receptor antagonist having nAChR antagonist activity is selected from the group consisting of amantadine and dextromethorphan, and pharmaceutically acceptable salts, hydrates and pharmaceutically active enantiomers thereof. Each possibility represents a separate embodiment of the invention.

In certain embodiments, the NMDA receptor antagonist also has dopaminergic activity. The term “dopaminergic activity” as used herein refers to the ability of the NMDA receptor antagonist to also modulate the activity of a receptor of the dopamine receptor family.

As used herein, the term “dopamine receptor family” refers to a group of proteins that function as receptors for dopamine. The group contains subset of proteins which are encoded by genes which exhibit homology of greater than 65% with each other in their deduced amino acid sequences within presumed transmembrane regions (linearly contiguous stretches of hydrophobic amino acids, bordered by charged or polar amino acids, that are long enough to form secondary protein structures that span a lipid bilayer). Three human dopamine receptor subfamilies are known to date: dopamine D₁ receptor, dopamine D₂ receptor and dopamine D₃ receptor.

In certain embodiments, the NMDA receptor antagonist is also a dopamine D₂ receptor agonist.

In certain embodiments, the NMDA receptor antagonist having dopamine D₂ receptor agonist activity is ketamine, or pharmaceutically acceptable salts, hydrates or pharmaceutically active enantiomers thereof. Each possibility represents a separate embodiment of the invention.

In certain embodiments, the NMDA receptor antagonist also has neuro-protective activity. The term “neuro-protective activity” as used herein refers to the effects of reducing or ameliorating nervous insult, and protecting or reviving neuronal cells that have suffered nervous insult. As used herein, the term “nervous insult” refers to any damage to neuronal cell or tissue resulting from various causes such as metabolic, toxic, neurotoxic and chemical causes. In certain embodiments, the NMDA receptor antagonist is also a sigma-1 receptor agonist.

As used herein, the term “sigma-1 receptor” refers to an expression product of the SIGMAR1 gene which encodes a chaperone protein at the endoplasmic reticulum (ER) that modulates calcium signaling through the IP3 receptor.

In certain embodiments, the NMDA receptor antagonist having sigma-1 receptor agonist activity is selected from the group consisting of L-687,384, amitriptyline and 1-benzyl-6′-methoxy-6′,7′-dihydrospiro[piperidine-4,4′-thieno[3.2-c]pyran], and pharmaceutically acceptable salts, hydrates and pharmaceutically active enantiomers thereof. Each possibility represents a separate embodiment of the invention.

The present invention also contemplates combination therapy comprising the NMDA receptor antagonist described herein with standard methods of treating GD. Non-limiting examples of standard methods of treating GD include various pain reduction therapies, blood transfusions, orthopedic surgery for bone and joint involvement, bone marrow transplantation, stem cell transplantation, splenectomy, gene therapy, enzyme replacement therapy (ERT), substrate reduction therapy (SRT), and pharmacological chaperone therapy agent.

According to specific embodiments, the NMDA receptor antagonist treatment is combined with an agent selected from the group consisting of an enzyme replacement therapy agent, substrate reduction therapy agent and pharmacological chaperone therapy agent.

As used herein “enzyme replacement therapy (ERT)” refers to the exogenous administration of glucocerebrosidase (GCD). A number of health regulatory agency-approved versions of GCD are available on the market. Examples include, but are not limited to, Elelyso (taliglucerase), Cerezyme (imiglucerase), Vpriv (velaglucerase) and Ceredase (alglucerase).

As used herein, the term “Substrate reduction therapy (SRT) agent” refers to an agent (e.g. small molecule) which inhibits the synthesis of the natural substrate of the GCD, glucosylceramide (or GL1). A number of health regulatory agency-approved versions of SRT are available on the market. Examples include, but are not limited to, Miglustat (Zavesca®) and Eliglustat Tartrate.

As used herein, the term “pharmacological chaperone therapy agent” (PCT) refers to an agent (e.g. small molecule) which can promote the correct folding and stabilize mutant forms of GCD, to thereby rescue the mutated enzyme from degradation presumably in the endoplasmic reticulum (ER) or in other cellular protein degradation/disposal systems and/or prevent the accumulation of misfolded protein in the cell. Pharmacological chaperones can be designed to cross the blood brain barrier (BBB) making them candidates for the treatment of neuronopathic forms of GD that are not responsive to ERT.

The NMDA antagonists and/or the agents of some embodiments of the invention can be administered to an organism per se, or in a pharmaceutical composition where it is mixed with suitable carriers or excipients.

The term “pharmaceutical composition” as used herein refers to any composition comprising at least one pharmaceutically active ingredient and at least one pharmaceutically acceptable carrier. The purpose of a pharmaceutical composition is to facilitate administration of a compound to an organism. Herein the term “active ingredient” refers to the NMDA receptor antagonist accountable for the biological effect.

Herein, the terms “pharmaceutically acceptable carrier” and a “physiologically acceptable carrier” which may be interchangeably used refer to a non-toxic solid, semisolid or liquid filler, carrier diluent or excipient of any type that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols. The term “pharmaceutically active enantiomer” as used herein refers to one or more stereoisomers of an indicated molecule, having an indicated biological activity or function. By way of example, an NMDA receptor antagonist may be used as a racemic mixture, or as enriched or purified (S) or (R) chirality drug. The relative activity of each stereoisomer may be determined using standard techniques known in the art.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

In certain embodiments, the NMDA receptor antagonist and/or the agents described above are formulated for oral delivery. In certain embodiments the NMDA receptor antagonist and/or the agents are formulated for sustained release. In certain embodiments, the NMDA receptor antagonist and/or the agents described above is formulated for injection.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method. Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (e.g. NMDA receptor antagonist) effective to prevent, alleviate or ameliorate symptoms of a disorder (e.g., GD, e.g. Type 3 GD) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide that levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

According to another aspect of the present invention there is provided a kit identified for use in treating neuropathic form of Gaucher Disease (GD), comprising a packaging material packaging an NMDA receptor antagonist and an agent selected from the group consisting of an enzyme replacement therapy agent, substrate reduction therapy agent and pharmacological chaperone therapy agent.

As further shown in Example 4 of the Examples section which follows the inventors have uncovered that the presence of guanine in a single nucleotide variation (SNP ID rs29869040) in a non-coding sequence of the Grin2b gene correlates with nGD, while the presence of adenine in this SNP correlates with relative CBE-insensitivity and more moderate phenotypes of GD; in addition Grin2b mRNA and protein expression levels correlate with severe nGD.

Thus, the teachings of the invention can be also used to diagnose neuropathic form of GD in a patient, by determining sequence variation in the Grin2B gene or amount of an expression product of the Grin2B gene.

Thus, according to an aspect of the present invention, there is provided a method of diagnosing neuropathic form of Gaucher Disease (GD) in a patient, the method comprising

determining in a biological sample of the patient a sequence variation that affects an amount of an expression product of a Grin2B gene and/or an amount of an expression product of a Grin2B gene, wherein presence of the sequence variation, an amount of the expression product above a predetermined level and/or increased amount of the expression product relative to a biological sample of a healthy patient or a patient diagnosed with GD type I is indicative of a neuropathic form of GD, thereby diagnosing neuropathic form of GD in the patient.

According to specific embodiments, the patient or subject is diagnosed with GD.

According to another aspect of the present invention, there is provided a method of treating a patient diagnosed with neuropathic form of Gaucher Disease (GD), the method comprising:

(a) diagnosing the patient according to the method of some embodiments of the invention; and wherein presence of the sequence variation and/or an amount of the expression product above a predetermined level and/or increased amount expression product relative to a biological sample of a healthy patient or a patient diagnosed with GD type I is indicated,

(b) treating the patient with a NMDA receptor antagonist, thereby treating the patient diagnosed with neuropathic form of GD.

To determine sequence variation, DNA is first obtained from a biological sample of the tested subject. Biological samples that can be used according to specific embodiments of the present invention include, but are not limited to, body fluids such as whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid and chorionic villi, and cell or tissue biopsy (e.g. brain biopsy).

Once the sample is obtained, DNA is extracted using methods which are well known in the art. As described herein above methods of detecting sequence variations are well known in the art and include, but not limited to, DNA sequencing, electrophoresis, an enzyme-based mismatch detection assay and a hybridization assay such as PCR, RT-PCR, RNase protection, in-situ hybridization, primer extension, Southern blot, Northern Blot and dot blot analysis. Sequence variations in a specific gene can also be determined at the protein level using e.g. chromatography, electrophoretic methods, immunodetection assays such as ELISA and western blot analysis and immunohistochemistry. According to specific embodiments, the sequence variation (e.g. SNP) is determined using genomic sequence analysis.

According to specific embodiments of the invention, detection of the sequence variation is performed by hybridizing the biological sample, the cell, or fractions or extracts thereof with a probe (e.g. oligonucleotide probe or primer) which specifically hybridizes with a polynucleotide comprising the sequence variation (e.g. SNP). Detailed description on probes that can be used in the present invention is further provided hereinbelow. According to specific embodiments, the hybridizing is effected under conditions which allow the formation of a complex comprising DNA comprising a sequence variation (e.g. SNP) present in the cell and the probe.

According to other specific embodiments of the invention, detection of the sequence variation is performed by contacting the biological sample, the cell, or fractions or extracts thereof with an antibody which specifically binds to a polypeptide comprising the sequence variation (e.g. SNP). According to specific embodiments, the contacting is effected under conditions which allow the formation of a complex comprising a polypeptide comprising the sequence variation (e.g. SNP) present in the cell and the antibody (i.e. immunocomplex).

The nucleotide/probe complex or immunocomplex can be formed at a variety of temperatures, salt concentration and pH values which may vary depending on the method and the biological sample used and those of skills in the art are capable of adjusting the conditions suitable for the formation of each complex.

According to specific embodiments, the sequence variation is an SNP.

According to specific embodiments, the sequence variation is in a non-coding sequence of the Grin2B gene.

As used herein, the phrases “level of expression” and “amount of expression” refers to the degree of gene expression and/or gene product activity in a biological sample. For example, up-regulation or down-regulation of various genes can affect the level of the gene product (i.e., RNA and/or protein).

It should be noted that the level of expression can be determined in arbitrary absolute units, or in normalized units (relative to known expression levels of a control reference). For example, when using DNA chips, the expression levels are normalized according to the chips' internal controls or by using quantile normalization such as RMA.

According to specific embodiments the amount of expression is determined using an RNA and/or a protein detection method.

According to specific embodiments the detection method is selected from the group consisting of PCR, oligonucleotide microarray, immunoprecipitation, Western blot analysis and FACS.

According to some embodiments of the invention, the RNA or protein molecules are extracted from the cell of the subject. Thus, according to specific embodiments, the method further comprises extracting a RNA or a protein from the cell prior to the comparing.

Methods of extracting RNA or protein molecules from cells of a subject are well known in the art. The extracted RNA can be further processed to a cDNA. Methods of and commercially available kits for converting RNA to cDNA are well known in the art and include e.g. the use of the enzyme reverse transcriptase. Once obtained, the RNA, cDNA or protein molecules can be characterized for the expression and/or activity level of various RNA, cDNA and/or protein molecules using methods known in the arts.

According to specific embodiment, the expression of the Grin2B gene can be determined at the nucleic acid level using RNA or DNA detection methods.

Thus, according to some embodiments of the invention, detection of the expression level of the RNA of the Grin2B pathway is performed by hybridizing the biological sample, the cell, or fractions or extracts thereof with a probe (e.g. oligonucleotide probe or primer) which specifically hybridizes with a polynucleotide expressed from the Grin2B gene (e.g., including any alternative spliced form which is known in the art). Such a probe can be at any size, such as a short polynucleotide (e.g., of 15-200 bases), an intermediate polynucleotide of 100-2000 bases and a long polynucleotide of more than 2000 bases.

The probe used by the present invention can be any directly or indirectly labeled RNA molecule [e.g., RNA oligonucleotide (e.g., of 17-50 bases), an in vitro transcribed RNA molecule], DNA molecule (e.g., oligonucleotide, e.g., 15-50 bases, cDNA molecule, genomic molecule) and/or an analogue thereof [e.g., peptide nucleic acid (PNA)] which is specific to the RNA transcript of the Grin2B gene. According to specific embodiments, the probe is bound to a detectable moiety.

Oligonucleotides designed according to the teachings of the present invention can be generated according to any oligonucleotide synthesis method known in the art such as enzymatic synthesis or solid phase synthesis.

According to specific embodiments, the hybridizing is effected under conditions which allow the formation of a complex comprising mRNA or cDNA of a Grin2B gene present in the cell and the probe. The complex can be formed at a variety of temperatures, salt concentration and pH values which may vary depending on the method and the biological sample used and those of skills in the art are capable of adjusting the conditions suitable for the formation of each nucleotide/probe complex.

Thus, according to an aspect of the present invention there is provided a composition of matter comprising a polynucleotide sample of a patient diagnosed with Gaucher Disease (GD), and an oligonucleotide capable of specifically hybridizing with a polynucleotide expressed from a Grin2B gene and optionally wherein said oligonucleotide is labeled.

Non-limiting examples of methods of detecting RNA and/or cDNA molecules in a cell sample include Northern blot analysis, RT-PCR [e.g., a semi-quantitative RT-PCR, quantitative RT-PCR using e.g., the Light Cycler™ (Roche)], RNA in situ hybridization (using e.g., DNA or RNA probes to hybridize RNA molecules present in the cells or tissue sections), in situ RT-PCR (e.g., as described in Nuovo G J, et al. Am J Surg Pathol. 1993, 17: 683-90; Komminoth P, et al. Pathol Res Pract. 1994, 190: 1017-25), and oligonucleotide microarray (e.g., by hybridization of polynucleotide sequences derived from a sample to oligonucleotides attached to a solid surface [e.g., a glass wafer) with addressable location, such as Affymetrix microarray (Affymetrix®, Santa Clara, Calif.)].

As mentioned, according to specific embodiments, the expression of the Grin2B gene can be determined at the amino acid level using protein detection methods.

Thus, according to some embodiments of the invention, detection of the expression level of the protein of the Grin2B is performed by contacting the biological sample, the cell, or fractions or extracts thereof with an antibody which specifically binds to a polypeptide expressed from the Grin2B gene of (e.g., including any variants thereof which is known in the art). According to specific embodiments, the contacting is effected under conditions which allow the formation of a complex comprising polypeptide of a Grin2B gene involved present in the cell and the antibody (i.e. immunocomplex).

The immunocomplex can be formed at a variety of temperatures, salt concentration and pH values which may vary depending on the method and the biological sample used and those of skills in the art are capable of adjusting the conditions suitable for the formation of each immunocomplex.

Thus, according to an aspect of the present invention there is provided a composition of matter comprising a composition of matter comprising a polypeptide sample of a patient diagnosed with Gaucher Disease (GD), and an antibody capable of specifically binding a polypeptide expressed from a Grin2B gene and optionally a secondary antibody.

Non-limiting examples of methods of detecting the level and/or activity of specific protein molecules in a cell sample include Enzyme linked immunosorbent assay (ELISA), Western blot analysis, immunoprecipitation (IP), radio-immunoassay (RIA), Fluorescence activated cell sorting (FACS), immunohistochemical analysis, in situ activity assay (using e.g., a chromogenic substrate applied on the cells containing an active enzyme), in vitro activity assays (in which the activity of a particular enzyme is measured in a protein mixture extracted from the cells) and molecular weight-based approach. For example, in case the detection of the expression level of a secreted protein is desired, ELISA assay may be performed on a sample of fluid obtained from the subject (e.g., serum), which contains cell-secreted content.

According to specific embodiments, the antibody used by the present invention can be any directly or indirectly labeled antibody. According to specific embodiments, the oligonucleotide is bound to a detectable moiety.

The detectable moiety used by some embodiments of the invention can be, but is not limited to a fluorescent chemical (fluorophore), a phosphorescent chemical, a chemiluminescent chemical, a radioactive isotope (such as ^{[125]}iodine), an enzyme, a fluorescent polypeptide, an affinity tag, and molecules (contrast agents) detectable by Positron Emission Tomagraphy (PET) or Magnetic Resonance Imaging (MRI).

As described above, the level of expression of Grin2B gene in a biological sample of the patient is compared to the level of expression of the at least one gene in a biological sample of a healthy patient or a patient diagnosed with GD type I.

As used herein, the term “healthy patient” refers to a patient not afflicted with GD.

As described above, presence of sequence variation that affects an amount of an expression product of a Grin2B gene, an amount of said expression product above a predetermined level and/or increased amount of said expression product relative to a biological sample of a healthy patient or a patient diagnosed with GD type I is indicative of a neuropathic form of GD.

As used herein the phrase “an amount above a predetermined threshold” or “increased amount” refers to at least a statistically significant upregulation with respect to the amount found in a similar biological sample of a healthy subject using the same method of quantification.

According to specific embodiments the increased amount is by at least 5%, at least 10%, at least 20%, at least 30%, at least 40%, at least 50%, at least 60%, at least 70%, at least 80%, at least 90%, at least 95%, at least 100% or more, higher than about 2 times, higher than about three times, higher than about four time, higher than about five times, higher than about six times, higher than about seven times, higher than about eight times, higher than about nine times, higher than about 20 times, higher than about 50 times, higher than about 100 times, higher than about 200 times, higher than about 350, higher than about 500 times, higher than about 1000 times, or more relative to the control sample.

It is expected that during the life of a patent maturing from this application many relevant NMDA receptor antagonists will be developed and the scope of the term “NMDA receptor antagonist” is intended to include all such new technologies a priori.

As used herein the term “about” refers to ±10%

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples. They should, in no way be construed, however, as limiting the broad scope of the invention.

### Examples

Reference is now made to the following examples, which together with the above descriptions illustrate some embodiments of the invention in a non limiting fashion. Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

Mice—Pure inbred mouse strains were obtained from the Jackson laboratories (USA), Harlan UK and Harlan Israel. Specifically, BTBR T⁺ Itpr3^{tf/J} C3H/HeJ, DBA/2J, MRL/MpJ, WSB/EiJ, MOLF/Ei, PWD/PhJ, KK/HiJ, BPL/1J, I/LnJ, BPN/3J, and LP/J were obtained from Jackson; A/J, AKR/J, NZW/LacJ, 129S1/SvImJ were obtained from Harlan laboratories in the UK; and C57BL/6JOlaHsd, BALB/cJ and FVB/NJ were obtained from the Harlan laboratories in Israel. Gba^{flox/flox}; nestin-Cre mice, a mouse model of GD were generated as described in Vitner et al. Nature Medicine (2014) 20: 204-208; and Enquist, I. B. et al. Proc. Natl. Acad. Sci. USA (2007) 104: 17483-17488. These mice recapitulate many features of human nGD, although their Gba1 deficiency is restricted to neural and macroglial lineages. Mice were maintained under specific pathogen-free conditions and handled according to protocols approved by the Weizmann Institute Animal Care Committee according to international guidelines.

CBE injections—Conduritol-β-epoxide (CBE) was obtained from Calbiochem Millipore (Darmstadt, Germany). CBE at a dose of 25 mg or 50 mg per kg body weight per day or PBS (control) were administered i.p. to mice daily starting from postnatal day 8 (P8) of age.

Treatment with NMDA receptor agonists and antagonists—All agonists and antagonists were administered daily from P8. D-cycloserine (Sigma) was administered daily at a dose of 200 mg/kg/day. MK-801 (Sigma) was administered daily at a dose of 0.3 mg/Kg/day. Memantine (Sigma) was administered daily at a dose of 3 or 30 mg/Kg/day. Ifenprodil (Sigma) was administered daily at a dose of 9 mg/Kg/day.

Immunohistochemistry—Mice were sacrificed and whole brains were fixed in 4% paraformaldehyde:PBS overnight at 4° C. Tissues were washed in PBS and sectioned with a microtome. Forty micrometer coronal sections were processed free floating in 2% BSA, 0.2% Triton X-100, PBS through all blocking, antibody incubation, and wash steps. Sections were incubated overnight at 4° C. with primary anti rat CD68 (AbD Serotec, Oxford, UK; 1:1000). Following washes, sections were incubated with secondary Cy2-conjugated donkey anti-rat antibody (1:200, Jackson). The presence of CD68-positive cells in cortical layer V of the brain was analyzed using an epifluorescent microscope (Zeiss).

RNA extraction and quantitative PCR Following treatment, mice were sacrificed and their brains removed, placed on a Young Mouse Brain Slicer Matrix (BSMYS001-1, Zivic instruments, Pittsburgh, Pa., USA) and cut into 1 mm coronal sections. The cerebral cortex was separated using a spatula and snap-frozen in liquid N₂ and stored at −80° C. Total RNA was isolated using the RNeasy mini kit (Qiagen GmbH, Hilden, Germany) according to manufacturer's instructions. Quantitative polymerase chain reaction (Q-PCR) was performed using PerfeCTa SYBR Green FastMix (Quanta BioSciences, Gaithersburg, Md., USA) and an ABI Prism 7300 Sequence Detection System (Applied Biosystems, Foster City, Calif., USA). The primer concentration was 13 nM in a reaction volume of 20 μl and cDNA equivalent to 2 ng of total RNA. Each reaction was performed in three biological replicates and four technical triplicates. The thermal cycling parameters were as follows: step 1, 95° C. for 10 minutes; step 2, 95° C. for 15 seconds, 60° C. for 30 seconds and 68° C. for 30 seconds. Step 2 was repeated for 40 cycles and was followed by a dissociation step. Fold-change in mRNA levels was calculated using the comparative cycle threshold method using TATA box binding protein (TBP) for normalization. P-values were calculated using a two-tailed, two-independent sample Student's t-test. Primers sequences were as follows:

Grin2b forward primer (SEQ ID NO: 2):
5′-CGCCCAGATCCTCGATTTCA-3′;
Grin2b reverse prime (SEQ ID NO: 3):
5′-CTGGAAGAACATGGAGGACTCA-3′.
Tbp forward primer (SEQ ID NO: 4):
5′-TGCTGTTGGTGATTGTTGGT-3′;
and
Tbp reverse primer (SEQ ID NO: 5):
5′-CTGGCTTGTGTGGGAAAGAT-3′;

GBA1 activity assay—GBA1 activity was performed as previously described (Farfel-Becker et al. Hum Mol Genet. 2014; 23(4):843-54). Briefly, frozen half-brain samples were sonicated in Mcllvaine's buffer (0.1 M citric acid, pH 4.2, 0.2 M Na₂HPO₄, 29:21, vol:vol). Tissue homogenates containing 50 μg of protein were incubated at 37° C. with 8 μM C6-NBD-GlcCer (Avanti Polar Lipids, Alabaster, Ala., USA) in a final volume of 25 μl Mcllvaine's buffer for 30 minutes. Lipids were extracted and the lower phase separated by thin layer chromatography using chloroform:methanol:9.8 mM CaCl2 (60:35:8, vol:vol:vol) as the developing solvent. C6-NBD-ceramide was identified with an authentic standard using a Typhoon 9410 variable mode imager and bands were quantified by Image-QuantTL (GE Healthcare, Chalfont St Giles, UK).

Sphingolipid analysis—LC-ESI-MS/MS was performed using an ABI 4000 quadrupole-linear ion trap mass spectrometer as previously described (Farfel-Becker et al. Hum Mol Genet. 2014; 23(4):843-54). The sphingolipid internal standards were obtained from Avanti Polar Lipids (Alabaster, Ala., USA).

Genome-Wide Association Studies—GWA mapping was performed on quantitative lifespan phenotypes by efficient mixed-model association (EMMA), using individual single-nucleotide polymorphism (SNP) associations as previously described (Kang et al, 2008). EMMA corrects for genetic relatedness and population structure, minimizing false associations. To assure high mapping resolution of the mouse genome, the association was performed using 4,000,000 SNPs/strain. The threshold for genome-wide significance after stringent Bonferroni correction was 1.25×10⁻⁸. Although it has been previously demonstrated that p<0.05 genome-wide equivalent for GWA using EMMA in the Hybrid mouse diversity panel (HMDP) is p=4.1×10⁻⁶ (Kang et al, 2008), to avoid false associations P values ≦1×10⁻⁷ were considered as significant. The Manhattan plot was generated using the R package (Turner S D. qqman: an R package for visualizing GWAS results using Q-Q and Manhattan plots. bioRxiv doi:://dx.doi.org/10.1101/005165).

Locomotor testing—Locomotor coordination was weekly evaluated in the treated strains by the hanging wire test, a validated test for LSD mouse models (Alvarez et al. 2008). Briefly, the mouse was placed with forepaws at a center of a horizontal bar (3 mm diameter; 35 mm long). The body position of the animal was observed for 30 seconds and scored as previously described (Alvarez et al. FASEB J. 2008; 22(10): 3617-27): 0=falls off within 10 seconds; 1, =hangs onto bar by two forepaws; 2=attempts to climb onto bar; 3=hangs onto bar by two forepaws plus one or both hindpaws; 4=hangs by all four paws plus tail wrapped around bar; and 5=active escape to the end of bar.

Human Samples and WB—Human brains samples were obtained with informed consent. Homogenates were prepared as described (Vitner et al 2014). Blots were incubated with anti NR2B (1:1000, Millipore, 06-600) and GAPDH (1:10,000, Chemicon, MAB374) antibodies.

Statistical analyses—All data are shown as mean±SEM. Comparisons between two samples were performed using a two-tailed Student's t-test. P<0.05 was considered statistically significant.

### Example 1. Selection of a Mouse Model for Neuropathic Gaucher Disease (nGD)

To study the potential of different mouse stains to serve as a reliable model for Type 2/3 GD, 15 different inbred mouse strains were chosen. CBE has been used to induce GD in mice for many years (Mumford et al., 1975), and it was used to generate the first animal model for GD disease. CBE is water soluble and crosses the blood brain barrier. The 15 mice strains chosen have diverse phylogenetic origins and their SNP profile is freely available (FIG. 1A). Mice were daily injected i.p. with 25 mg/kg/day of CBE starting on postnatal day 8 and for the rest of their lives (Vitner et al., 2014). The survival of the 15 mice strains was followed for over 200 days, as summarized in FIG. 1B and Table 2 below. The results obtained show that each mouse strain reacts differently to GD induction by CBE. The strains could be divided into two groups: mice in the first group were very homogeneous in their behavior; they developed a very aggressive disease with a short life span. The second group of strains presented a much longer life expectancy with a broader spectrum of phenotypes. Among each strain, the life span variability was low, suggesting that the response to CBE treatment in each strain is a consequence of the genetic background.

To further characterize the effect of CBE treatment on the different strains, motor behavior was followed weekly using the hanging wire test, starting at postnatal day 21 for a period of 2 months. The hanging wire test evaluates coordination and locomotor function, wherein a high score means better coordination. As shown in FIG. 1C, the short living CBE-treated strains also showed poor motor behaviors starting at very early time points; while the long living strains presented higher motor behavior values. Some strains which presented long life spans, such as SV129 and BALB/c, showed poor motor behavior at later stages, while others such as KK/HIJ had high motor scores at all time points tested. Independently of the strain, the PBS-treated mice showed high hanging wire test scores, indicating that the differences in motor behavior resulted as a consequence of CBE treatment (FIG. 1C). Of note, strains PWD/PhJ and WSB/EiJ were excluded in the motor behavior analysis due to their wild behavior.

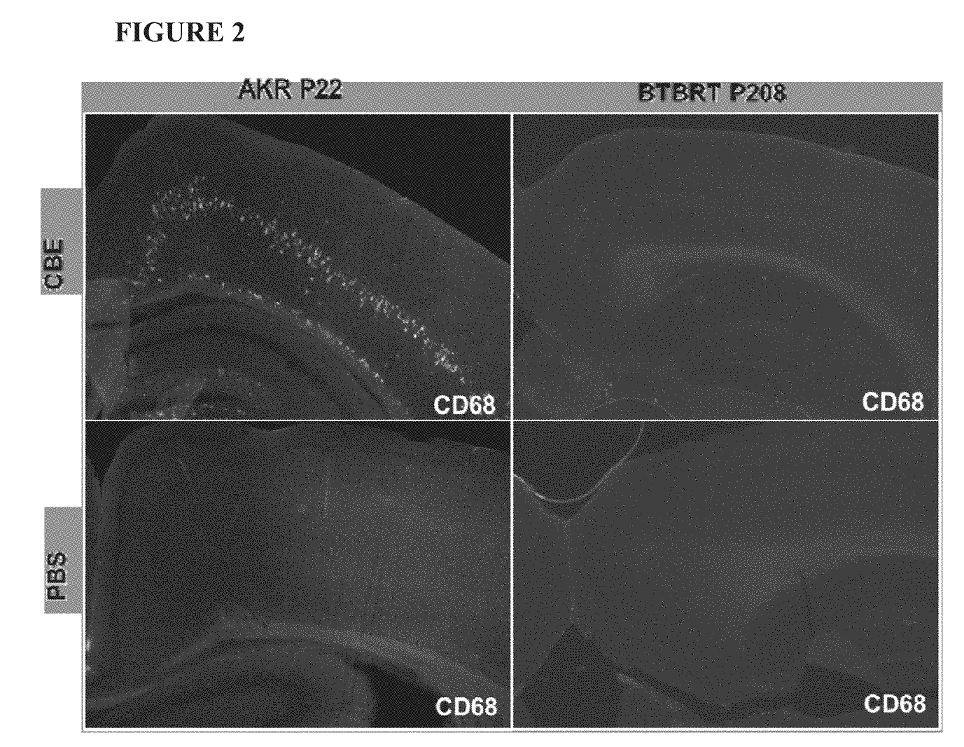

To verify the results of Example 1, the presence of CD68-positive cells in the cortical layer V of the brain was analyzed in two exemplary strains, AKR/J (average mortality on day 24) and BTBR T^{+ITPR3TF/J} (100% mortality on day 199). As is illustrated in FIG. 2, the AKR/J mice which are more prone to mortality by CBE also demonstrated overt brain pathology indicating that they can be used as a good model for Type 2 and/or 3 GD, while the BTBR T^{+ITPR3TF/J} mice are less sensitive to CBE both in terms of mortality and brain pathology.

Taken together, of the strains tested, A/J, AKR/J, C3H/HeJ, DBA/2J, MRL/MpJ, C57BL/6J, WSB/EiJ and MOLF/EiJ were found to be most sensitive to CBE, providing a good mouse model for Type 2/3 GD.

### Example 2. GBA1 Activity and Lipid Levels in the Brains of the Different Mouse Strains do not Correlate with the Differences in Survival Rates

To eliminate the possibility that the phenotypic differences observed in the different mouse strains were caused by differential CBE metabolism in the brain, GBA1 enzymatic activity in mice treated for 10 days with CBE or PBS (control) was measured. As evident in FIG. 3A, CBE significantly reduced, to the same extent, cerebral GBA1 activity in all strains tested. Overall, the remaining GBA1 activity was 5-10% of the controls (FIG. 3A). There was no correlation between GBA1 activity in the brain and life spans neither in the PBS(R²=0.126, data not shown) nor CBE conditions (R²=0.0581, FIG. 3B). In addition, no correlation was found between inhibition of GBA1 activity and life span when evaluated according to strains (R²=0.0085, data not shown).

In the next step, the consequences of GBA1 inhibition, GlcCer accumulation, was analyzed. As expected, CBE-treated mice presented higher levels of GlcCer in the brain in comparison to the controls. The average elevation in GlcCer levels in CBE-treated brains was 3.7-fold compared to PBS-treated mice (FIG. 3C). C18, the most abundant GlcCer species in the brain, was the main GlcCer species elevated (not shown). Importantly, there was no correlation between the levels of GlcCer in the brain and life spans either in the PBS(R²=0.1441, data not shown) or CBE conditions (R²=0.0569, FIG. 3C). There was also no correlation between the change in GlcCer levels in each strain and the life spans (R²=0.0311, data not shown). In addition, no correlation was found between each individual GlcCer subspecies (C14, C16, C18:1, C18, C20, C22, C24:1, C24, C26:1, and C26) and life spans under PBS and CBE conditions (data not shown).

Glucosylsphingosine (GlcSo) is another lipid known to be elevated in GD and it has been proposed as a GD biomarker (Rolfs et al. PLoS One. 2013 Nov. 20; 8(11):e79732). An average elevation of ˜36-fold in GlcSo levels was found in the CBE-treated brains as compared to the controls (FIG. 3D). The potential correlation between GlcSo levels and the life spans of mice was explored—no correlation was found either under PBS(R²=0.068, data not shown), or in the CBE-treated brains (R²=0.0013, FIG. 3D). Similarly to the other lipids, there was no correlation between the change in GlcSo levels and mice life spans (R²=0.0775, data not shown).

In addition, GalCer and GalSo levels were analyzed in the brains of the treated mice (FIGS. 3E-3F). CBE-treated mice showed no elevation in these lipids and there was no correlation between lipid levels and mice life spans (R²=0.0975 and 6×10⁻⁵ for GalCer and GalSo respectively).

Taken together, the lack of correlation between GBA1 activity or lipids levels and mice life span indicates that the phenotypic differences observed in the tested strains are not due to differential metabolism of CBE or differential accumulation of levels of GlcCer or GlcSPh, indicating involvement of modifier genes.

### Example 3. Genome Wide Association Study

To discover novel genes involved in GD progression, a Genome Wide Association Study (GWAS) was performed for the 15 inbred strains mentioned above. GWAS in model organisms has great potential to identify risk factors for complex traits related to human diseases (Frazeret al., 2007; Peters et al., 2007). Model organism-association mapping is potentially more powerful than human association mapping because it is possible to reduce the effect of environmental factors by replicating phenotype measurements in genetically identical organisms (Peters et al., 2007). Moreover, the information generated in many of the ongoing genotyping projects in model organisms is freely-available, allowing in-silico mapping of complex traits in model organisms. For example, recent advances in genomic sequence analysis and SNP discovery have led to the availability of dense SNP maps for a large number of inbred strains of mice (Kirby et al 2010). These mice SNP profiles are a rich resource for mapping of complex traits and have been widely used for GWAS mapping (Liu et al 2007).

To avoid potential false associations due to the genetic-relatedness among strains, a novel bioinformatics tool based on Efficient Mixed-Model Association (EMMA) was used. EMMA is a statistical test for model organism-association mapping that corrects the confounding effect from population structure and genetic relatedness (Kang et al., 2008). Another way to avoid false associations due to population structure is to choose mouse strains from different phylogenetic origins (Clarke et al., 2011).

4,000,000 SNPs/strain were used to allow good coverage of the genome. FIGS. 4A-B shows a Manhattan plot indicating the log of the odds of association of the markers in each chromosome. The GWAS analysis identified Grin2b (p value=6.58E⁻⁰⁸), the gene encoding for the subunit B of the NMDA glutamate receptor (NMDAR2B or NR2B), as a potential modifier gene of GD progression. Specifically, a single nucleotide variation was identified (SNP ID rs29869040) on chromosome 6 (Chr. position 136080931), in a non-coding sequence of the Grin2b gene (located in the intron region of Grin2b mRNA), where guanine correlated with CBE-sensitivity and a phenotype of early GD, while adenine correlated with relative CBE-insensitivity and more moderate phenotypes of GD (association p value=1.73×10⁻¹⁰).

The average survival time and Grin2b SNP variation of the 15 mice strains tested are summarized in Table 2 below.

TABLE 2

| SNP ID | Average survival | |
|---|---|---|
| rs29869040 | time (days) | Mouse strain |
| G | 23 | A/J |
| G | 24 | AKR/J |
| G | 24 | C3H/HeJ |
| G | 26 | DBA/2J |
| G | 29 | C57BL6/JolaHsd |
| G | 30 | MRL/MpJ |
| G | 38 | WSB/EiJ |
| G | 41 | MOLF/Ei |
| A | 84 | NZW/LacJ |
| A | 106 | FVB/NJ |
| A | 108 | 129S1/SvImJ |
| G | 119 | PWD/Ph |
| A | 130 | BALB/cJ |
| A | 143 | KK/HlJ |
| A | 199 | BTBR T^{+ITPR3TF/J} |

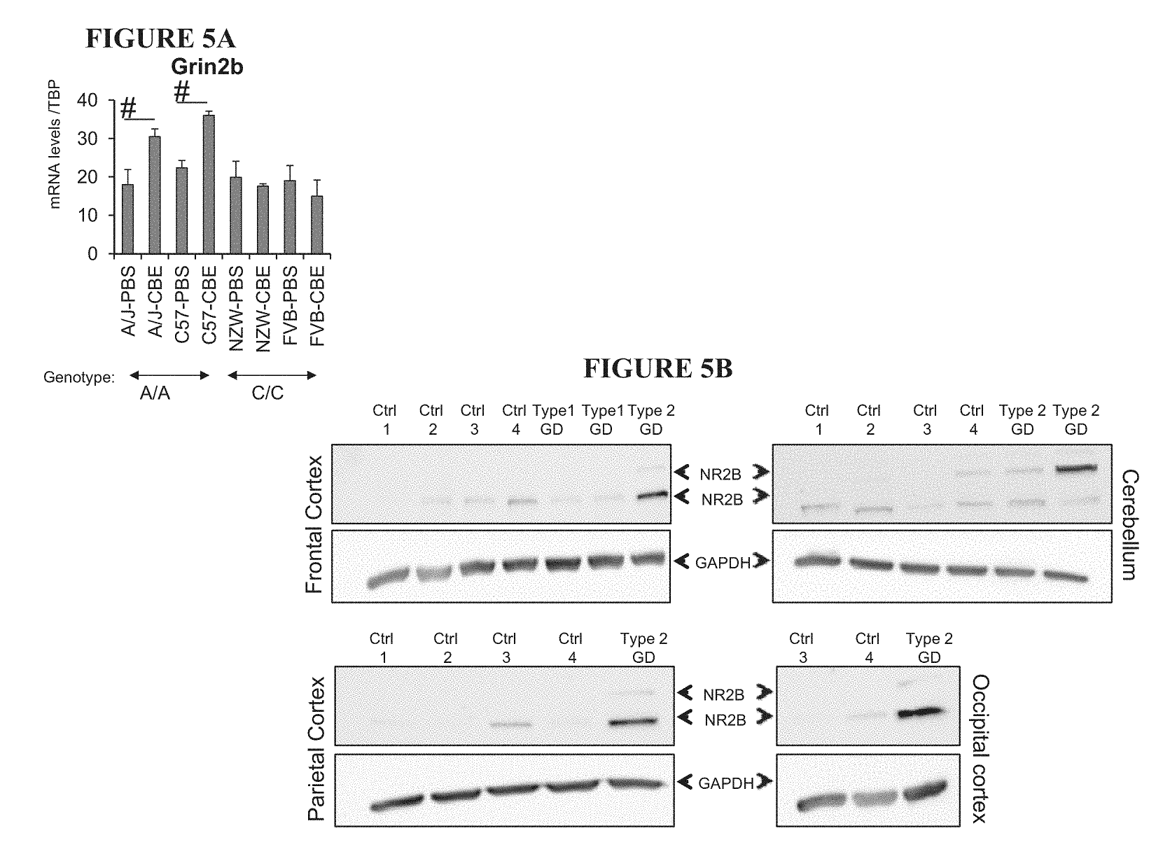

Based on the predictive studies described above, the involvement of Grin2b in the pathogenesis of neuropathic GD was evaluated. The significant associated SNP was found in intron. Since non-coding SNPs can regulate the expression of quantitative trait locus (QTL), the mRNA levels of Grin2b were determined by qPCR. The samples used were taken from cerebral cortex because Grin2b is expressed in the brain and this area demonstrated the pathology. The transcript levels was determined in 2 short living (A/J and C57BL6/JolaHsd) and 2 long living strains (NZW and FVB) 10 days following PBS or CBE treatment. Different pattern of gene expression was found—Grin2b expression levels were increased only in the short-living strains following CBE-treatments, indicating a regulation of the expression levels under stress conditions (FIG. 5A).

Furthermore, the levels of NR2B protein in postmortem biopsies of a pathogenic brain area, frontal cortex, in controls, Type 1 and 2 GD patients were evaluated by western blot. Analogously to the qPCR results obtained in the mice tissues, no changes in the levels of NR2B in the tissues of Type 1 GD patients was detected, while elevated levels in the neuropathic patient were observed, suggesting that NR2B plays a pathological role in nGD (FIG. 5B).

In the past, one of the inventors of the present invention have shown that GlcCer accumulation induces changes in functional calcium stores (Korkotian et al., 1999, Pelled at al., 2005). In addition, the Inventors demonstrated that rat hippocampal neurons treated with CBE are more sensitive to the toxic effects of high concentrations of glutamate in vitro (Pelled at al., 2000), suggesting hyper-activation of glutamate receptors. Altogether, this suggests that the present GWAS analysis is likely to have revealed an important candidate gene that may be involved in the modification of GD pathological cascades; and that agonists of NMDA receptor in the long living strains should reduce their life spans and antagonists of the NMDA receptor should increase the life spans of the short living strains.

### Example 4. The Effect of NMDA Antagonist MK-801 vs. NMDA Agonist D-Cycloserine on nGD Progression

To study the biological relevance of glutamate excitotoxicity in neuropathic Gaucher Disease (GD), a pharmacological approach was used. First, the role of a potent NMDA antagonist, MK-801, in GD pathogenesis, was studied. MK-801 is one of the most specific NMDA blockers that exist. It binds the ion channel of the receptor in a non-competitive manner at several domains, preventing the influx of Ca²⁺ through the channel. MK-801 has been extensively studied for the treatment of animal models of neurological diseases (Lipton, 2006). However, it antagonizes both synaptic and extra-synaptic NMDA receptors (Parsons and Raymond, 2014).

FIG. 6 illustrates how blocking NMDA signaling with MK-801 (0.3 mg/Kg/day) extended the survival of CBE-treated (25 mg/Kg/day) C3H mice, suggesting that the NMDA receptor is a good therapeutic target for anti-GD agents.

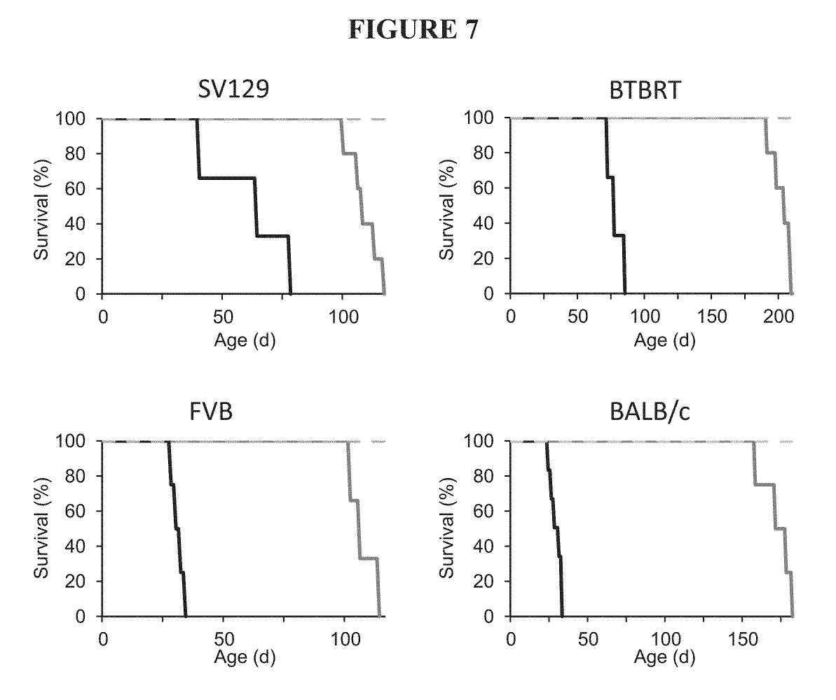

On the contrary, a potent NMDA agonist, D-cycloserine, administered to 4 long living mouse strains (BALB/cJ, FVB/NJ, 129S1/SvImJ, and BTBR T⁺ Itpr3^{tf/J}) reduced the life spans of the CBE-treated mice (FIG. 7).

### Example 5. Examples for Other NMDA Antagonists for the Treatment of nGD

Despite the positive effects of MK-801 in extending the lifespan of GD mice, it cannot be used to treat patients due to its high affinity to the receptor and low off-rate kinetics, blocking not just the toxic effects of excessive calcium influx via extra-synaptic receptors, but also crucial normal functions of glutamate signaling (Lipton, 2004). Therefore, memantine, an uncompetitive, open-channel blocker that presents a faster off-rate binding kinetics to the receptor than MK-801 and preferentially blocks extra-synaptic NMDA receptors (Lipton, 2006, Parsons and Raymond, 2014), was tested. Memantine is less specific than MK-801. It acts as a non-competitive antagonist at the 5-HT₃ receptor and alpha-7 nAChR (Rammes et al., 2001; Aracava et al., 2005) and acts as an agonist to the dopamine D₂ receptor (Seeman et al., 2008). Memantine is approved by the U.S. FDA and the EMA for treatment of moderate-to-severe Alzheimer's disease (Reisberg et al., 2003) and dementia with Lewy bodies (Aarsland et al., 2009).

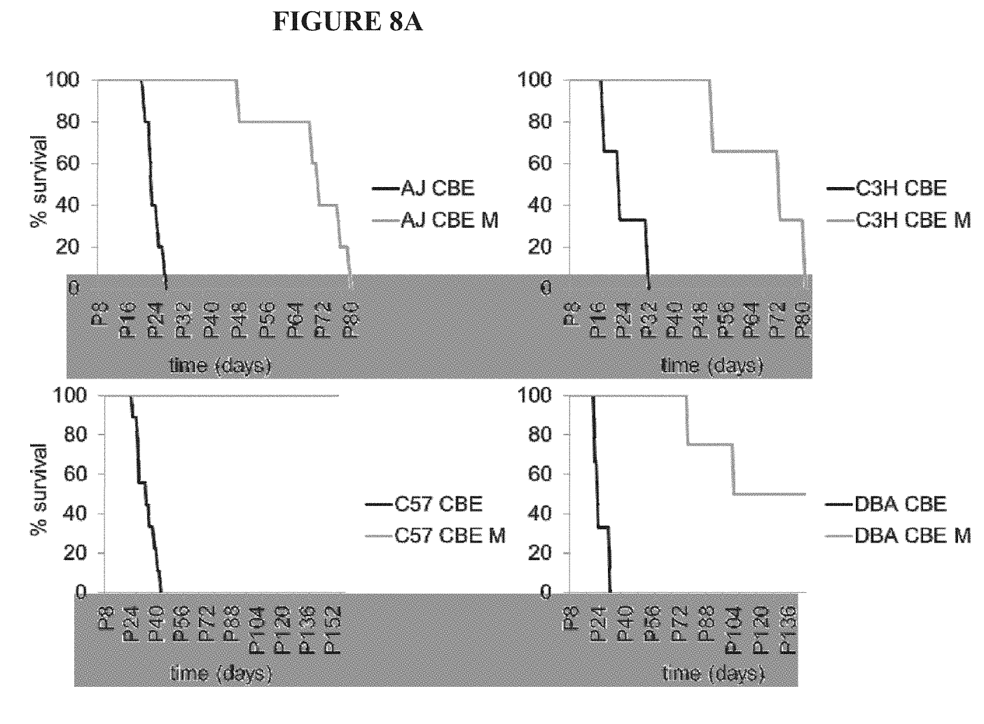

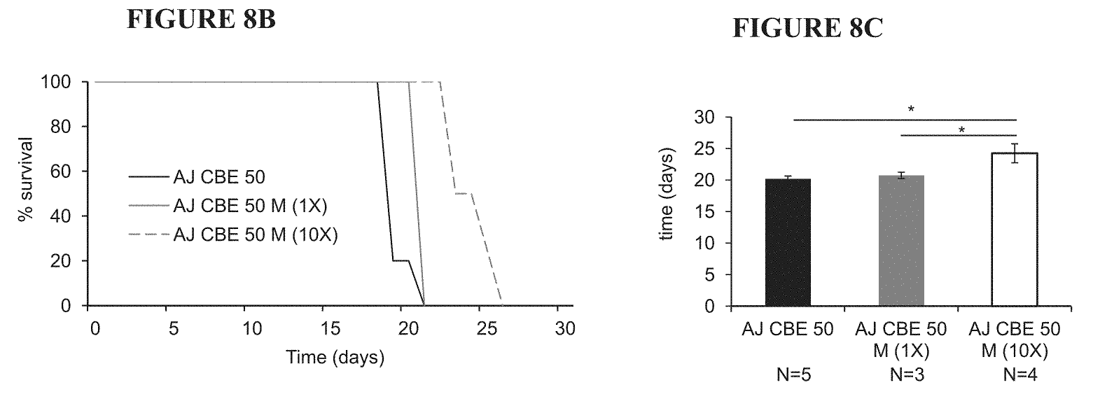

Several mouse strains [A/J (AJ), C3H/HeJ (C3H), DBA/2J (DBA) and C57BL6/JolaHsd (C57)] that develop neuropathic GD using CBE as shown in FIG. 1B and Table 2 above were treated with CBE (25 mg/kg day) or with CBE plus memantine (3 mg/kg day). As shown in FIG. 8A, in all tested strains treatment with memantine significantly extended the life span of the mice.

Memantine treatment at a higher dose of 30 mg/kg/day significantly extended survival of AJ mice even at a higher CBE treatment dose of 50 mg/kg/day (FIGS. 8B-C).

Treatment with memantine increased the life span of the neuropathic GD mice to a greater extent than MK-801, possibly because memantine blocks preferentially the extra-synaptic NMDA receptors while MK-801 blocks both the synaptic and extra-synaptic, NMDA receptors (Parsons and Raymond, 2014).

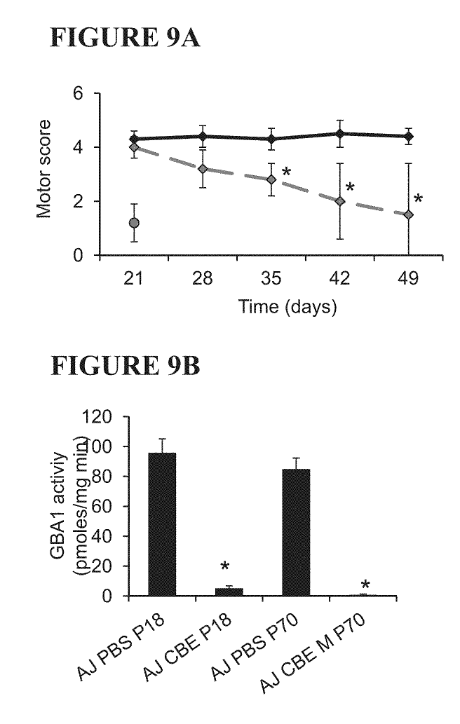

In a similar manner, treatment with memantine delayed the progression of the motor dysfunction induced by CBE, as assessed by the hanging wire test (FIG. 9A). At postnatal day 49, the memantine-treated mice presented the same score as CBE-treated mice at P21, suggesting that memantine is a potential drug to treat the most aggressive forms of nGD.

Importantly, treatment with memantine did not interfere with GBA1 inhibition in the brain, which was almost undetectable at postnatal day 70 (P70), the last stages of the mouse disease (FIG. 9B).

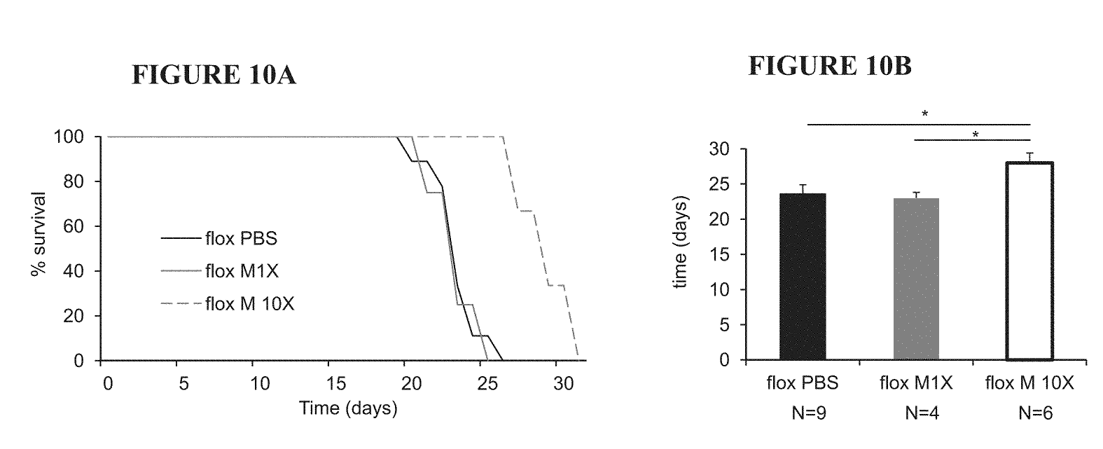

Since CBE can inhibit other enzymes besides GBA1, the effects of memantine were further evaluated in a genetic mouse model of nGD, where the GBA1 deletion occurs in neurons and astroglial cells. Remarkably, as shown in FIGS. 10A-B, memantine treatment significantly extended survival of nGD mice

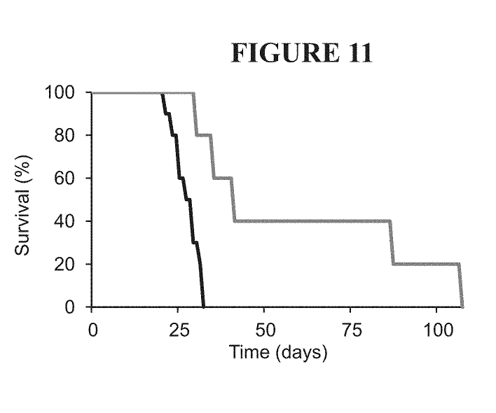

In the last stage, another NMDA receptor antagonist, Ifenprodil, was tested in the CBE-treatment mouse model. In accordance with the other MK801 and memantine, this drug also showed increments in the life spans of the CBE-treated mice (FIG. 11).

Taken together, the data indicate that NMDA receptor antagonists may serve as a novel therapeutic target for nGD.

### REFERENCES (Other References are Cited Throughout the Application)

Aarsland D., Ballard C., Walker Z., Bostrom F., Alves G., Kossakowski K., Leroi, I. Pozo-Rodriguez F., Minthon L., Londos E., Memantine in patients with Parkinson's disease dementia or dementia with Lewy bodies: a double-blind, placebo-controlled, multicentre trial. Lancet, Neurology, 8, 613-618 (2009).
Aracava Y., Pereira E. F., Maelicke A., Albuquerque E. X. Memantine blocks alpha7* nicotinic acetylcholine receptors more potently than n-methyl-D-aspartate receptors in rat hippocampal neurons. J. Pharmacol. Exp. Ther., 312, 1195-1205 (2005).
Beavan M. S. and Schapira A. H. V., Glucocerebrosidase mutations and the pathogenesis of Parkinson disease. Annals of Medicine, 45, 511-521 (2013).
Bliss T. V. and Collingridge, G. L., A synaptic model of memory: Long-term potentiation in the hippocampus. Nature, 361, 31-39 (1993).
Caudle W. M., Zhang J., Glutamate, excitotoxicity, and programmed cell death in Parkinson disease. Exp. Neurol., 220, 230-233 (2009).
Clarke G. M. et al., Basic statistical analysis in genetic case-control studies. Nat. Protoc., 6, 121-133 (2011).
Cooke S. F. and Bliss T. V., Plasticity in the human central nervous system. Brain, 129, 1659-1673 (2006).
Costa J., Gomes C., de Carvalho M., Diagnosis, pathogenesis and therapeutic targets in amyotrophic lateral sclerosis. CNS Neurol. Disord. Drug Targets, 9, 764-778 (2010).
Cox T. M., Gaucher Disease: clinical profile and therapeutic developments. Biologics, 4, 299-313 (2010).
Deegan P. B. and Cox T. M., Imiglucerase in the treatment of Gaucher disease: a history and perspective. Drug Design, Development and Therapy, 6, 81-106 (2012).
Essa M. M., Braidy N., Vijayan K. R., Subash S., Guillemin G. J. Excitotoxicity in the pathogenesis of autism. Neurotox. Res., 23, 393-400 (2013).
Frazer K. A. et al., A sequence-based variation map of 8.27 million SNPs in inbred mouse strains. Nature, 448, 1050-1053 (2007).
Fuller M., Meikle P. J. and Hopwood J J., Epidemiology of lysosomal storage diseases: an overview. Fabry Disease: Perspectives from 5 Years of FOS (2006).
Gonzalez D. E et al., Enzyme replacement therapy with velaglucerase alfa in Gaucher disease: Results from a randomized, double-blind, multinational, Phase 3 study. Am. J. Hematol., 88, 166-171 (2013).
Hollak C. E. M., An evidence-based review of the potential benefits of taliglucerase alfa in the treatment of patients with Gaucher disease. Core Evidence, 7, 15-20 (2012).
Kang H. M. et al., Efficient control of population structure in model organism association mapping. Genetics, 178, 1709-1723 (2008).
Korkotian E., Schwarz A., Pelled D., Schwarzmann G., Segal M., Futerman A. H., Elevation of intracellular glucosylceramide levels results in an increase in endoplasmic reticulum density and in functional calcium stores in cultured neurons. J. Biol. Chem., 274, 21673-21678 (1999).
Kostandy B. B., The role of glutamate in neuronal ischemic injury: the role of spark in fire. Neurol. Sci., 33, 223-37 (2012).
Lipton S. A., Failures and successes of NMDA receptor antagonists: molecular basis for the use of open-channel blockers like memantine in the treatment of acute and chronic neurologic insults. NeuroRx., 1, 101-110 (2004).
Lipton S. A., Paradigm shift in neuroprotection by NMDA receptor blockade: memantine and beyond. Nat. Rev. Drug Discov., 5, 160-70 (2006).
Liu X. B. et al., Switching of NMDA receptor 2A and 2B subunits at thalamic and cortical synapses during early postnatal development. The Journal of Neuroscience, 24, 40, 8885-8895 (2004).
Machaczka M. et al., Substrate reduction therapy with miglustat for Type 1 Gaucher disease: A retrospective analysis from a single institution. Upsala Journal of Medical Sciences, 117, 28-34 (2012).
Mehta A., Prabhakar M., Kumar P., Deshmukh R., Sharma P. L., Excitotoxicity: bridge to various triggers in neurodegenerative disorders. Eur. J. Pharmacol., 698, 6-18 (2013).
Mumford R. A. et al., The Gaucher mouse. Biochem. Biophys. Res. Commun., 67, 1, 85-90 (1975).
Nicholls J. G., Martin A. R., Fuchs P. A., Brown D. A., Diamond M. E., Weisblat D., (2012). From neuron to brain (5th ed.). Sunderland, Mass.: Sinauer Associates.
Parsons M. P., Raymond L. A. Extrasynaptic NMDA receptor involvement in central nervous system disorders. Neuron, 82, 279-293 (2014).
Pelled D., Shogomori H., Futerman A. H., The increased sensitivity of neurons with elevated glucocerebroside to neurotoxic agents can be reversed by Imiglucerase. J. Inh. Met. Dis., 23, 175-184 (2000).
Pelled D., Trajkovic-Bodennec S., Lloyd-Evans E., Sidransky E., Schiffmann R., Futerman A. H. Enhanced calcium release in the acute neuropathic form of Gaucher Disease. Neurobiol. Dis., 18, 83-88 (2005).
Peters L. L. et al., The mouse as a model for human biology: a resource guide for complex trait analysis. Nat. Rev. Genet., 8, 58-69 (2007).
Rammes G., Rupprecht R., Ferrari U., Zieglgänsberger W., Parsons C. G., The N-methyl-D-aspartate receptor channel blockers memantine, MRZ 2/579 and other amino-alkyl-cyclohexanes antagonise 5-HT(3) receptor currents in cultured HEK-293 and N1E-115 cell systems in a non-competitive manner. Neurosci. Lett., 306, 81-84 (2001).
Ratjen F. and Doring G., Cystic fibrosis. Lancet, 361, 681-689 (2003).
Reisberg B., Doody R., Stöffler A., Schmitt F., Ferris S., Möbius H. J., Memantine Study Group. Memantine in moderate-to-severe Alzheimer's disease. New Engl. J. Med., 348, 1333-1341 (2003).
Seeman P., Caruso C., Lasaga M., Memantine agonist action at dopamine D2 High receptors. Synapse, 62, 149-153 (2008).
Sidransky E. et al., Multicenter Analysis of Glucocerebrosidase Mutations in Parkinson's Disease, N. Engl. J. Med. 361, 1651-1661 (2009).
Traynelis S. F., Wollmuth L. P., Mcbain C. J., Menniti F. S., Vance K. M., Ogden K. K., Hansen K. B., Yuan H., Myers S. J. and Dingledine R., Glutamate receptor ion channels: Structure, regulation, and function. Pharmacological Reviews, 62, 405-496 (2010).
Vitner et al., RIPK3 as a potential therapeutic target for Gaucher's disease. Nat. Med., 20, 2, 204-208 (2014).
Walker F. O., Huntington's disease. Lancet, 369, 218-228 (2007).
Zhang C. K. et al., Genome-wide association study of N370S homozygous Gaucher disease reveals the candidacy of CLN8 gene as a genetic modifier contributing to extreme phenotypic variation. Am. J. Hematol., 87, 4, 377-383 (2012).

1. (canceled)

2. A method for treating a neuropathic form of Gaucher Disease (GD) in a patient in need thereof, comprising administering a therapeutically effective amount of an NMDA receptor antagonist to said patient, thereby treating said neuropathic form of GD.

3-4. (canceled)

5. The method of claim 2, comprising administering to said patient a therapeutically effective amount of an agent selected from the group consisting of an enzyme replacement therapy agent, substrate reduction therapy agent and pharmacological chaperone therapy agent.

6. A kit identified for use in treating neuropathic form of Gaucher Disease (GD), comprising a packaging material packaging an NMDA receptor antagonist and an agent selected from the group consisting of an enzyme replacement therapy agent, substrate reduction therapy agent and pharmacological chaperone therapy agent.

7. The method of claim 2, wherein said NMDA receptor is an extra-synaptic NMDA receptor.

8. The method of claim 2, wherein said NMDA receptor comprises an NR2B subunit.

9. The method of claim 2, wherein said NMDA receptor antagonist is selected from the group consisting of memantine, nitromemantine, neramexane, ketamine, amantadine, dextromethorphan, L-687,384, amitriptyline, 1-benzyl-6′-methoxy-6′,7′-dihydrospiro[piperidine-4,4′-thieno[3.2-c]pyran], eliprodil, ifenprodil, orphenadrine, kynurenic acid, felbamate, and pharmaceutically acceptable salts, hydrates and pharmaceutically active enantiomers thereof.

10. The method of claim 2, wherein said NMDA receptor antagonist is selected from the group consisting of memantine, nitromemantine, neramexane, and pharmaceutically acceptable salts, hydrates and pharmaceutically active enantiomers thereof.

11. The method of claim 2, wherein said NMDA receptor antagonist is selected from the group consisting of memantine, eliprodil and ifenprodil or a pharmaceutically acceptable salt, hydrate or pharmaceutically active enantiomer thereof.

12. The method of claim 2, wherein said NMDA receptor antagonist is memantine (3,5-dimethyl-1-adamantanamine) or a pharmaceutically acceptable salt, hydrate or pharmaceutically active enantiomer thereof.

13. The method of claim 2, wherein said NMDA receptor antagonist also has serotonergic activity; and/or wherein said NMDA receptor antagonist is also a 5-HT₃ receptor antagonist.

14. (canceled)

15. The method of claim 2, wherein said NMDA receptor antagonist also has cholinergic activity; and/or wherein said NMDA receptor antagonist is also a nicotinic acetylcholine receptor (nAChR) antagonist, and/or wherein said NMDA receptor antagonist is selected from the group consisting of amantadine and dextromethorphan, and pharmaceutically acceptable salts, hydrates and pharmaceutically active enantiomers thereof.

16-17. (canceled)

18. The method of claim 2, wherein said NMDA receptor antagonist also has dopaminergic activity; and/or wherein said NMDA receptor antagonist is also a dopamine D₂ receptor agonist.

19. (canceled)

20. The method of claim 2, wherein said NMDA receptor antagonist is also a sigma-1 receptor agonist; and/or wherein said NMDA receptor antagonist is selected from the group consisting of L-687,384, amitriptyline and 1-benzyl-6′-methoxy-6′,7′-dihydrospiro[piperidine-4,4′-thieno[3.2-c]pyran], and pharmaceutically acceptable salts, hydrates and pharmaceutically active enantiomers thereof.

21. (canceled)

22. The method of claim 2, wherein said NMDA receptor antagonist has neuro-protective activity.

23. The method of claim 2, wherein said GD is sub-acute, chronic neuropathic GD Type 3.

24. The method of claim 23, wherein said GD Type 3 is GD Type 3a.

25. The method of claim 23, wherein said GD Type 3 is GD Type 3b.

26. The method of claim 2, wherein said NMDA receptor antagonist is formulated for oral delivery.

27. The method of claim 2, wherein said NMDA receptor antagonist is formulated for injection.

28-36. (canceled)

37. A method of treating a patient diagnosed with neuropathic form of Gaucher Disease (GD), the method comprising:
(a) determining in a biological sample of the patient a sequence variation that affects an amount of an expression product of a Grin2B gene and/or an amount of an expression product of a Grin2B gene; and wherein presence of said sequence variation and/or an amount of said expression product above a predetermined level and/or increased amount expression product relative to a biological sample of a healthy patient or a patient diagnosed with GD type I is indicated,
(b) treating said patient with a NMDA receptor antagonist,
thereby treating the patient diagnosed with neuropathic form of GD.
